(19) United States
(12) Patent Application Publication
(10) Pub. No.: US 2019/0247449 A1
(43) Pub. Date: Aug. 15, 2019

(54) COMPOSITIONS COMPRISING A NON-PATHOGENIC BACTERIA AND METHODS FOR PROTECTING PLANT AND ANIMAL HOSTS FROM FUNGAL, BACTERIAL AND VIRAL DISEASES

(71) Applicant: GRACE BREEDING LTD., Kfar Saba (IL)

(72) Inventors: Amit AVIDOV, Kiryat Tivon (IL); Avner BARAZANI, Omer (IL); Mor ZEILKHA, Ramat Gan (IL)

(21) Appl. No.: 16/332,959

(22) PCT Filed: Sep. 13, 2017
(86) PCT No.: PCT/IL2017/051038

§ 371 Date: Mar. 13, 2019

Related U.S. Application Data
(60) Provisional application No. 62/394,229, filed on Sep. 14, 2016.

(51) Int. Cl. A61K 35/747; A23K 10/18; A61K 35/745; A61K 36/8905; A61K 36/28; A23K 50/90; A23K 20/121; A23K 20/163; A23K 20/111

(52) U.S. Cl. CPC A61K 35/747; A23K 10/18; A61K 35/745; A61K 36/8905; A23K 20/111; A23K 50/90; A23K 20/121; A23K 20/163; A61K 36/28

(57) ABSTRACT

The present invention is directed to a method for preventing and/or treating infection of plant or animal host species by fungal, bacterial and/or viral pathogens, wherein said method comprises the steps of providing a mixture of one or more non-pathogenic bacteria and one or more activating agents; and administering said mixture to said host species. The invention also encompasses compositions comprising a mixture of non-pathogenic bacteria and one or more activating agents.

### FIELD OF THE INVENTION

The present invention relates to the use of non-pathogenic bacterial species together with one or more activating agents in the prevention and/or treatment of microbial disease in a range of different biological hosts.

### BACKGROUND OF THE INVENTION

The use of non-pathogenic soil-borne bacteria for protecting plant and other host species of agricultural and horticultural interest against bacterial and fungal attack is well known in the art. One example of a non-pathogenic bacterial species used in such systems is Bacillus subtilis.

There are several prior art publications describing fungicidal and bactericidal products comprising Bacillus subtilis, as well as the use of such products in the protection of agriculturally-important plants against microbial diseases. However, these products mainly have low levels of activity, and as a result have been used primarily as adjuncts for increasing the activity of conventional chemical agents. In addition, the literature also describes the use of such products as the sole agents for the protection of organically-cultivated plants (i.e. in situations in which the use of more powerful chemical agents would not be permitted).

Thus, while the use of biological control of microbial pests using Bacillus subtilis is highly advantageous due to its low toxicity to host cells, its relatively low-level activity has prevented this approach from being more widely adopted in agriculture and horticulture on a larger, commercial scale.

There is therefore an unmet need for compositions and methods for the protection of commercially-important plant and animal species that combine the low host toxicity of systems based on the use of non-pathogenic bacteria such as Bacillus subtilis with greatly increased effectiveness in enhancing the ability of the host species to resist microbial attack.

The present invention meets this need.

### SUMMARY OF THE INVENTION

The present invention is primarily directed to a method for increasing the ability of a plant or animal host species to resist damage caused by fungal, bacterial and/or viral pathogens.

In its most general form, this method comprises the steps of:
a) Providing a mixture of non-pathogenic bacteria and one or more activating agents; and
b) Administering the mixture of step (a) to a plant or animal host species.

In certain cases, the two components mentioned in step (a), above (i.e. the non-pathogenic bacteria and the activating agent(s)) may be administered separately. Consequently, in such cases, the method for increasing the ability of plant or animal host species to resist damage caused by fungal, bacterial and/or viral pathogens comprises the steps of:

a) providing separately:
(i) a composition comprising one or more non-pathogenic bacteria; and
(ii) a composition comprising one or more activating agents; and
   b) separately administering each of compositions (i) and (ii) to said host species.

Without wishing to be bound by theory, it is to be noted that the protective effect seen with the method and composition of the present invention may be due to a direct anti-microbial activity, enhancement of the host species' resistance to microbial infection (e.g. by enhancing the host immune response) or to a combination of both mechanisms.

Consequently, the abovementioned phrase “ability to resist damage caused by fungal, bacterial and/or viral pathogens” should be understood in terms of one or more of enhanced survival, size and health of the host species (and where relevant, increased yield of an agricultural or horticultural product) in the presence of microbial pathogens, regardless of the actual mechanism(s) involved in the enhancement of these parameters. Similarly, the term “damage caused by fungal, bacterial and/or viral pathogens” should be understood as any detrimental effect on the host species that are directly or indirectly related to the presence of the microbial pathogens within the cells or tissues of said host species, or in close proximity to said host species. These detrimental effects include (but are not limited to) interference with growth, size or reproductive capability of the host species, fatal and non-fatal lesions on one or more organs of the host species, decreased yield of products of agricultural importance (e.g. fruit, vegetables, pulses, milk, honey etc.) produced by the host species.

In the context of the present invention, the term “activating agent” is used to denote a substance which when present in a mixture together with the non-pathogenic bacteria or when delivered separately therefrom, is capable of enhancing the beneficial effects of said non-pathogenic bacterial cells on the treated plant or animal host species. This enhancement may, in some cases, be a result of a synergistic interaction between the non-pathogenic bacteria and the activating agents. In the alternative, the activating agents and the non-pathogenic bacteria may each be devoid of any significant beneficial effect on the host when used alone, but may cause significant anti-microbial, immunostimulatory and/or other beneficial effects in the host species when the two classes of substance are administered together or consecutively.

The present inventors have unexpectedly found that many of the activating agents suitable for use in the method of the present invention share a common feature, namely their ability to inhibit inflammatory mediators that are more generally associated with higher animal species (such as Tumor Necrosis Factor alpha [TNF-α]) rather than with plant species. Thus, in one preferred embodiment of the present invention, the one or more activating agents are substances having anti-inflammatory activity.

In one aspect, the present invention is directed to a method for preventing and/or treating infection of a plant or animal host species by fungal, bacterial and/or viral pathogens, wherein said method comprises the steps of:
a) Providing a mixture of non-pathogenic bacteria and one or more activating agents; and
b) Administering the mixture of step (a) to said host species.

In an alternative embodiment of this aspect of the invention, the non-pathogenic bacterial and the activating agents may be administered separately, as explained hereinabove.

In a further aspect, the present invention is also directed to a method for increasing either the yield of a plant of agricultural or horticultural importance or the yield of a product (e.g. milk or honey) from an animal of agricultural importance, by means of:
a) Providing a mixture of non-pathogenic bacteria and one or more activating agents; and
b) Administering the mixture of step (a) to said host species.

In an alternative embodiment of this aspect of the invention, the non-pathogenic bacteria and the activating agents may be administered separately, as explained hereinabove.

It was disclosed hereinabove that in some implementations of the various methods of the present invention, the non-pathogenic bacteria and the activating agents may be administered separately, that is, one after the other. In such implementations, the first composition to be administered may be either the composition comprising the non-pathogenic bacteria or the composition comprising the one or more activating agents. In certain other embodiments of the type in which the non-pathogenic bacteria and the activating agents are administered separately, both of them are administered to the host species at approximately the same time.

In another aspect, the present invention also provides a composition comprising a mixture of non-pathogenic bacteria and one or more activating agents, wherein said one or more activating agents are substances having anti-inflammatory activity, as will be discussed in further detail hereinbelow.

Many different species and strains of non-pathogenic bacteria may be used in combination with the activating agents described herein (or alternatively, may be administered separately and sequentially). The term ‘non-pathogenic’ is used in this context to indicate that the selected species have no, or very few, toxic or other deleterious effects on the host species to which the composition of the invention containing the bacteria are being administered.

In one preferred embodiment of the methods and compositions defined herein, the non-pathogenic bacteria are selected from the group consisting of Bacillus subtilis and probiotic bacteria.

In one preferred embodiment, the non-pathogenic bacterial species is Bacillus subtilis.

Several different strains of this species may be used. However, in one highly preferred embodiment, the strain used is QST 713.

In another preferred embodiment, the non-pathogenic bacteria are selected from one or more species of probiotic bacteria. It is to be noted, in this regard, that the term ‘probiotic bacteria’ for the present purposes is to be understood to refer to live microorganisms that are believed to provide health benefits when consumed or otherwise administered to the intended host. Many such probiotic bacteria are known, with many of them being species of the genus Bifidobacterium (e.g. B. longum and B. breve) or the genus Lactobacillus (e.g. L. rhamnosus, L. casei, L. helveticus and so on). In one preferred embodiment, the probiotic bacteria used in the compositions and methods of the present invention are selected from the group consisting of L. rhamnosus, L. Casei, L. Plantarum, L. helveticus (acidophilus), B. Longum, B. breve, Pediococcus Acidilactici, Lactococcus lactis and combinations thereof.

Further embodiments and advantages of the invention will become apparent as the description proceeds.

### BRIEF DESCRIPTION OF THE DRAWINGS

FIG. 1 graphically presents results from an initial screening of phytochemicals for their potential use as activating agents for B. subtilis for enhancement of anti-bacterial and anti-fungal effects.

FIG. 2 presents results for B. subtilis activation of combinations of 3 or 4 different activating agent combinations and for the fungicidal and bactericidal activities of compositions containing those combinations together with B. subtilis.

FIG. 3 presents results of investigations similar to those presented in FIG. 2, except that the activating agents are used at a different concentration.

FIG. 4 presents results for fungicidal and bactericidal activity using compositions similar to those used to generate the results shown in FIG. 1, except that a different formulation of B. subtilis was use.

FIG. 5 presents fungicidal and bactericidal results obtained using compositions similar those used to generate the results of FIG. 5, except that the activating agents were used at a different concentration.

FIG. 6 presents results showing the fungicidal and bactericidal effects of combinations of the extracts of two plants—Aster tataricus and Cyperus rotundus with B. subtilis.

FIG. 7 presents results of studies similar to those which generated the results of FIG. 6, except that the two plant extracts were used at a different concentration.

FIG. 8 demonstrates that inoculation of cucumber seedlings with different combinations of activating agents and B. subtilis is capable of protecting cucumber plants from both fungal and bacterial infections.

FIG. 9 presents results showing that inoculation of tomato seedlings with different combinations of activating agents and B. subtilis is able to protect tomato plants against microbial infection.

FIG. 10 presents results showing the increased survival of tomato plants infected with tomato mosaic virus, following treatment with compositions of the present invention.

FIG. 11 demonstrates the protective effect of compositions of the present invention against microbial infection in pepper plants, following inoculation of pepper plant seedlings with said compositions.

FIG. 12 demonstrates the protective effect of compositions of the present invention against microbial infection in maize, following inoculation with said compositions.

FIG. 13 presents results showing the protective effect of compositions of the present invention against microbial infection in wheat, following inoculation of seedlings with said compositions.

FIG. 14 demonstrates the protective effect of compositions of the present invention against microbial infection in rice plants, following inoculation of rice seedlings with said compositions.

FIG. 15 presents the results of a field study which demonstrate protection against antimicrobial infection in chickpea plants, following treatment with compositions of the present invention.

FIG. 16 presents in vitro results showing the almost complete elimination of Streptococcus sobrinus (a bacterial species of significance for the development of dental caries in humans) following treatment with compositions of the present invention.

FIG. 17 presents in vitro results showing the almost complete elimination of Lactobacilli (a species of significance for the development of dental caries in humans) following treatment with compositions of the present invention.

FIG. 18 is a trial map showing the division of a field into the different treatment groups used in a field study of the effect of compositions of the present invention on growing carrots and carrot plant foliage following infection with Candidatus liberibacter.

FIG. 19 graphically depicts the beneficial effects of a composition of the present invention in reducing the percentage of carrot plants having foliar lesions caused by Candidatus liberibacter.

FIG. 20 graphically depicts the beneficial effects of a composition of the present invention in reducing the average number of carrots having lesions caused by Candidatus liberibacter.

FIG. 21 presents data showing the increased number of bee larvae present in beehives following treatment of the bees with a composition of the present invention, as compared with untreated control.

FIG. 22 presents data showing the increased number of adult bees present in beehives following treatment of the bees with a composition of the present invention, as compared with untreated control.

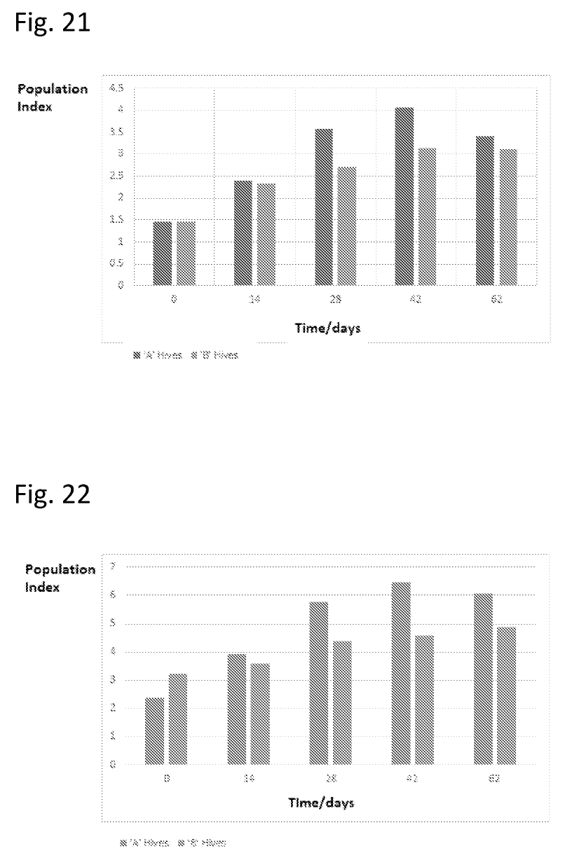

### DETAILED DESCRIPTION OF PREFERRED EMBODIMENTS

As explained hereinabove, the present inventors have unexpectedly found that many of the activating agents suitable for use in the method of the present invention (in combination with non-pathogenic bacteria such as B. subtilis or probiotic species) share the ability to inhibit inflammatory mediators that are more generally associated with higher animal species (such as Tumor Necrosis Factor alpha [TNF-α]). Thus, in one preferred embodiment of the present invention, the one or more activating agents are substances having anti-inflammatory activity.

As mentioned, it has been found by the present inventors that the aforementioned anti-inflammatory activity that is associated with the activating agents of the present invention is mediated, at least in part, by the inhibition of one or more key inflammatory mediators such as TNF-α and/or nitric oxide (NO). Consequently, in one preferred embodiment of the present invention, the one or more activating agents used in the aforementioned method are substances capable of inhibiting the production of NO and/or TNF-α.

In one further preferred embodiment of the present invention, the activating agents each have an IC₅₀ for the inhibition of NO production of less than 1.5 mg/ml and/or an IC₅₀ for the inhibition of TNF-α production of less than 2.5 mg/ml.

In another preferred embodiment, each individual activating agents (whether used alone or in combination with other such agents) has an IC₅₀ for the inhibition of NO production of less than 0.1 mg/ml and/or an IC₅₀ for the inhibition of TNF-α production of less than 0.2 mg/ml.

In a still further preferred embodiment, each individual activating agents (whether used alone or in combination with other such agents) has an IC₅₀ for the inhibition of NO production of less than 0.05 mg/ml and/or an IC₅₀ for the inhibition of TNF-α production of less than 0.1 mg/ml.

It is to be noted that the use of the IC₅₀ value (i.e. the concentration of an agent which causes 50% of the maximal inhibition of a mediator, agonist or other biologically active molecule) as a means for comparing the potency of antagonists and other biologically- and pharmacologically-active molecules, is well-known to all skilled-artisans in this field. Briefly, the IC₅₀ values may be obtained by plotting dose-response curves for a parameter such as inhibition of a particular inflammatory mediator, and extracting said values from said curves.

In another preferred embodiment, the activating agents are selected from the group consisting of Sclareol, Naringin, Nootkatone, Steviol glycoside and cannabidiol and combinations thereof.

In a yet further preferred embodiment, the activating agents (including those having the qualitative and quantitative anti-inflammatory properties disclosed above) are derived from plant material (such as crude plant extracts, such as whole plant aqueous extracts, partially purified or fractionated extracts, purified extracts and synthetic analogues of active molecules present in said extracts).

In one preferred embodiment of this aspect of the invention, the plant-derived activating agents are herbal extracts selected from the group consisting of Aster tataricus, Cyperus rotundus and combinations thereof.

In one preferred embodiment, the host species is a plant species, including (but not limited to) vegetables, pulses, grains, tropical species (such as bananas), sub-tropical species (such as citrus fruits), other trees and shrubs, flowering plants of horticultural interest, and so on.

In another preferred embodiment, the host species is an animal species, in particular an insect species of agricultural importance, such as various species of bees, including but not limited to honey bees (Apis mellifera. L). In another preferred embodiment, the animal species treated are mammals, including both human subjects and non-human species. With regard to the latter, many domesticated animals, or animals of agricultural importance may be treated with the compositions and methods of the present invention. In one preferred embodiment, the mammalian subject to be treated is a cow or sheep.

In one preferred embodiment, the non-pathogenic bacterial species is Bacillus subtilis.

Although many different strains of Bacillus subtilis may be used to perform the method of the present invention, in one preferred embodiment, the strain used is the QST 713 strain.

This strain may be obtained commercially in various different formulations, including Serenade® ASO and Cease®.

In another preferred embodiment, the non-pathogenic bacteria are selected from one or more species of probiotic bacteria. As explained hereinabove, many different species of probiotic bacteria (including, but not limited to, species of the Lactobacillus and Bifidobacterium genii may be used to perform the method of the present invention.

In some embodiments, between one and five of the above-mentioned activating agents is used to prepare the mixture used in step (a) of the method. In one preferred embodiment, all five of said activating agents is used.

In another particularly preferred embodiment, the five activating agents are present in the mixture in the following percentage ranges:

| | 98% active material | 98% active material |
|---|---|---|
| | minimal | Maximal |
| The material | concentration | concentration |
| Sclareol | 0.05% | 10% |
| Naringin | 0.05% | 10% |
| Nootkatone | 0.05% | 30% |
| Steviol glycoside | 0.20% | 80% |
| Cannabidiol | 0.01% | 10% |

Preferably, the percentage composition of the Bacillus subtilis cells and the activating agents in the mixture is as follows:
Bacillus subtilis (Serenade) 0.1%-10% (more preferably 0.5%-5%)
Activating agents 0.01%-10% (more preferably 0.05%-5%)

In embodiments of the methods disclosed and claimed herein in which the host species are plants, many different means of bringing the mixture of step (a) into contact with the host organism may be employed in step (b) of said method. These means include (but are not limited to): fertigation, spraying, emulsion, controlled release membranes or substrates, and combinations thereof.

In some preferred embodiments, the one or more non-pathogenic bacteria, one or more activating agents and/or combinations thereof are administered to the plant by means of foliar administration. This may be achieved, for example, by means of spraying these substances using conventional means.

In other preferred embodiments, the one or more non-pathogenic bacteria, one or more activating agents and/or combinations thereof are administered to the plant by means of adding these substances to the medium in which said plant is growing. This may be achieved by means of preparing granules or other substrates (such as absorbent fibers, pellets, beadlets etc.) which have been coated with the non-pathogenic bacteria and/or activating agents, or alternatively been caused to absorb these substances into their internal structure by immersion therein or by any other means. In one particularly preferred embodiment, the delivery form used comprises a plurality of granules (e.g. Perlite granules) which have been coated with the substances to be delivered. Generally (but not always), these granules may further comprise a release-control polymer, which is usually present as an exterior coating on the granule surface.

In a further preferred embodiment, the non-pathogenic bacteria, one or more activating agents and/or combinations thereof are administered by means of coating seeds of the plant species with these substances prior to sowing said seeds. Such coated seeds may further comprise one or more release-control polymers, generally (but not exclusively) in the form of an exterior coating.

In some embodiments, the method of the present invention comprises the separate administration of the non-pathogenic bacteria and the activating agents. Such separate administration may also be accomplished by any of the administration routes used for the mixtures of said non-pathogenic bacteria and activating agents described herein.

In some embodiments, the mixture of step (a) is administered to the host organisms to be treated in a continuous manner, for periods of between a few hours and about 180 days.

When the emulsion method is used, the treatment period is generally a few hours and a second treatment may be administered after about 10 days.

When a controlled release membrane or substrate is used, the treatment will take about 180 days. The controlled release substrate may be of several different types. In one preferred embodiment, this substrate is formed into granules, such as Perlite granules, as are well known to the skilled artisan in this technical field. Other options for control release substrates include various pellets, beads, micro-beads, fibers having a water absorbing capacity above 1:15 in relation to their dry weight. In order to achieve the desired controlled-release characteristics, the substrates may be coated with wax, Ethocel, other release-control polymers (as well known in the agricultural, pesticidal and pharmaceutical fields) and plant oils.

In the case that the method of the invention is used to treat animals, the composition of the present invention may be formulated for topical use in the form of gels or creams. Alternatively, the composition may be formulated such that it can be added to a solid or liquid animal feed that is already being administered to the animal on a daily basis. Finally, other dosage forms intended for oral or parenteral administration into animal host species will be well known to the skilled artisan in this field, and are all included within the scope of the present invention.

The present invention also provides a composition comprising a mixture of non-pathogenic bacteria and one or more activating agents, wherein said one or more activating agents are substances having anti-inflammatory activity. Preferably, such anti-inflammatory agents are capable of inhibiting the production or release of the anti-inflammatory mediators nitric oxide (NO) and/or TNF-α.

In one preferred embodiment of this aspect, said activating agents each have an IC₅₀ for the inhibition of NO production of less than 1.5 mg/ml and/or an IC₅₀ for the inhibition of TNF-α production of less than 2.5 mg/ml.

In another preferred embodiment, said activating agents each have an IC₅₀ for the inhibition of NO production of less than 0.1 mg/ml and/or an IC₅₀ for the inhibition of TNF-α production of less than 0.2 mg/ml.

In a still preferred embodiment, said activating agents each have an IC₅₀ for the inhibition of NO production of less than 0.05 mg/ml and/or an IC₅₀ for the inhibition of TNF-α production of less than 0.1 mg/ml.

In one preferred embodiment of this aspect, the present invention is directed to a plant-protection or animal-protection composition comprising a mixture of Bacillus subtilis and one or more activating agents selected from the group consisting of Sclareol, Naringin, Nootkatone, Steviol glycoside and cannabidiol.

In one preferred embodiment, any of the above-disclosed compositions may further comprise one or more additional components, including penetrating agents, stabilizers, solvents, sequestrants, emulsifiers and release-control (e.g. slow release) agents.

Examples of suitable penetrating agents—polar aprotic solvents DMSO, DMSO-d6, Dimethylformamide (DUF).

Examples of suitable non-ionic surfactant include Triton X-100, Tergitol 15-S-3, 15-S-5, 15-S-7.

Examples of suitable sequestrants include sodium phosphates, sodium gluconate, calcium chloride, potassium gluconate.

Examples of emulsifiers include polyaldo10-6-O, E-471, E-475, and E-476.

Examples of controlled release agents include coatings comprising dicyclopentadiene and linseed oil or a soy bean oil alkyd (e.g. the commercially-available coating composition sold under the registered trademark “Osmocote®”, and distributed by ICL Specialty Fertilizers, Israel, and disclosed in U.S. Pat. No. 4,657,576), and the polymer E603 obtainable from Sekisui Specialty Chemicals, Japan.

Of course, the additional components listed above are given only for the sake of illustration, and many other different additives and excipients may also be included in the compositions disclosed herein.

It is to be noted that in some of the preferred embodiments of the present invention, the mixture of activating agents includes both hydrophilic and hydrophobic substances. As a result, it is in many cases necessary to prepare the composition as an emulsified mixture of two separate components: an aqueous portion containing the more water-soluble agents dissolved in water and a hydrophobic portion containing the less water-soluble agents dissolved in fatty acids, medium chain triglycerides, ethanol, other solvents and combinations thereof.

In one preferred embodiment of the above-disclosed composition of the present invention, the non-pathogenic bacteria are selected from the group consisting of Bacillus subtilis and probiotic bacteria.

In one highly preferred embodiment, the non-pathogenic bacteria are bacteria of the species Bacillus subtilis. Although many different strains of this species may be used, in one preferred embodiment, the composition comprises the QST 713 strain.

In another highly preferred embodiment, the non-pathogenic bacteria in the composition are one or more species of probiotic bacteria. In one implementation of this embodiment, the probiotic bacteria are selected from the group consisting of L. rhamnosus, L. Casei, L. Plantarum, L. helveticus (acidophilus), B. Longum, B. breve, Pediococcus Acidilactici, Lactococcus lactis and combinations thereof.

In a further aspect, the present invention also provides a mixture of agents capable of causing activation of Bacillus subtilis, selected from the group consisting of: Sclareol, Naringin, Nootkatone, Steviol glycoside and cannabidiol, and combinations thereof.

A further advantage of the method and composition of the present invention is the positive influence that the mixture of non-pathogenic bacteria (such as, for example, B. subtilis and probiotic bacteria) and activating agents may have on the vigor of plants treated therewith, particularly during the early stages of seedling development. Consequently, in another aspect, the present invention is directed to a method for increasing the yield of a plant of agricultural or horticultural importance by means of:

a) providing a mixture of one or more non-pathogenic bacteria and one or more activating agents; and
b) administering the mixture of step (a) to said host species.

In another embodiment of this aspect, the invention also provides a method for increasing the yield of a plant of agricultural or horticultural importance by means of:

a) providing separately:
(i) a composition comprising one or more non-pathogenic bacteria; and
(ii) a composition comprising one or more activating agents; and
   b) separately administering each of compositions (i) and (ii) to said host species.

Similarly, the present invention also encompasses a method for increasing the yield of a product (for example: milk, honey etc.) from an animal of agricultural importance by means of:

a) providing separately:
(i) a composition comprising one or more non-pathogenic bacteria; and
(ii) a composition comprising one or more activating agents; and
   b) separately administering each of compositions (i) and (ii) to said host species.

In a variant of this method (as disclosed hereinabove in relation to other methods of the present invention), the non-pathogenic bacteria and composition comprising one or more activating agents may be administered separately to the host species.

The above-defined methods for increasing the yields of agricultural products and for increasing the ability of a plant or host species to resist microbial-induced damage, may each comprise any of the technical features that were disclosed and described hereinabove in connection with the method for preventing and/or treating infection of plant or animal host species by microbial pathogens.

In another aspect, the present invention is directed to a mixture of one or more non-pathogenic bacteria and one or more activating agents for use in the prevention or treatment of infection caused by fungal, bacterial and/or viral pathogens in animal species. In another aspect, the present invention is also directed to the use of a mixture of one or more non-pathogenic bacteria and one or more activating agents for the treatment and/or prevention of infection caused by fungal, bacterial and/or viral pathogens in plant or animal host species.

All of the technical features disclosed and described hereinabove in connection with the various methods of treatment, apply equally to the use of the mixture of non-pathogenic bacteria and activating agents disclosed immediately hereinabove.

The present invention will now be further illustrated with reference to the following non-limiting working examples and accompanying figures.

### EXAMPLES

### Material and Methods:

### 1. Non-Pathogenic Bacteria

a) Bacillus subtilis

For the purpose of the studies reported herein as Examples 1 to 18, the commercially-available QST 713 strain was used. This strain was obtained from the Bayer Corporation in two different formulations: 1) Serenade® ASO; and 2) Cease®. In most of the working examples presented below, Serenade® ASO was used as the source of Bacillus subtilis. However, in Examples 3 and 11, Cease® was used in place of Serenade®.

### b) Probiotic Bacteria

For the purpose of the studies reported herein as Examples 19 and 20, the commercially-available probiotic mixture known as ‘Jarro Dophilus’ was used to prepare the compositions of the present invention. Further details of this probiotic mixture are found in Example 19, hereinbelow.

### 2. Activating Agents

Following initial screening of a large number of candidate molecules, the following phytochemicals were selected for use as activating agents in the first part of the present study:

1. Sclareol—di-terpene alcohol extracted from Salvia sclarea.

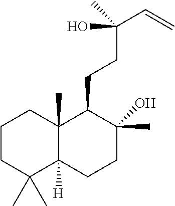 [SMILES: C=C[C@](C)(O)CC[C@@H]1[C@@]2(C)CCCC(C)(C)[C@@H]2CC[C@@]1(C)O]

2. Naringin—flavanone-7-O-glycoside extracted from grapefruit rind.

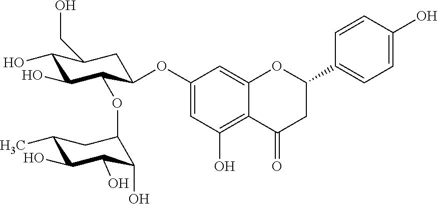

3. Nootkatone—sesquiterpene—extracted from orange rind.

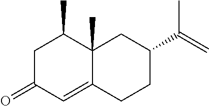 [SMILES: C=C(C)[C@@H]1CCC2=CC(=O)C[C@@H](C)[C@]2(C)C1]

4. Steviol glycoside—extracted from Stevia rebaudiana.

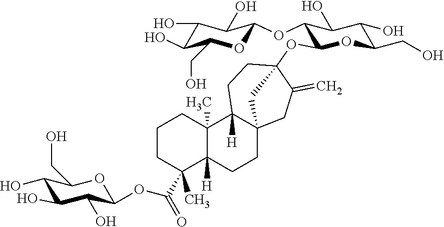

5. CBD—cannabidiol—extracted from hemp.

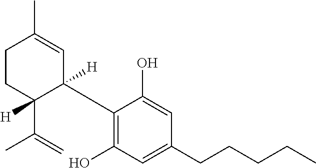 [SMILES: C=C(C)[C@@H]1CCC(C)=C[C@H]1c1c(O)cc(CCCCC)cc1O]

Following initial testing of combinations of five or less of these substances as activating agents for B. subtilis, the efficacy of additional herbal materials was also investigated, as described in Examples 4 and 5, hereinbelow.

### Example 1

### Initial Screening of Phytochemicals for their Potential Use as Activating Agents for Bacillus subtilis

### Introduction:

Cucumber (Cucumis sativus L) seedlings are highly susceptible to fungal and bacterial pathogens attacking the seedling during the germination process, and were therefore selected as a model plant to screen and calibrate the Bacillus subtilis and the phytochemicals that can cause activation thereof.

### Material and Methods:

### 1. Phytochemical Screening:

The potential phytochemicals were added to a mixture of 30 cc glucose 50% V/V substrate, 10 cc cocktail of fungal pathogens and 10 cc cocktail of bacterial pathogens in a Petri dish. The fungal cocktail contained: Botrytis cinerea, Rhizoctonia solani, Pythium spp. and non-pathogenic fungi used for the fermentation of tomatoes. The bacterial cocktail contained: Clavibacter michiganensis, Xanthomonas campestris, Pseudomonas syringae and non-pathogenic bacteria used for the fermentation of tomatoes.

Approximately 1000 potential phytochemicals were screened for their ability to activate Bacillus subtilis, by means of calculating a colony forming index for each test (0=no colony; 5=maximal colony size). The five phytochemicals listed above in the introduction to the Examples section were selected from the approximately 1000 phytochemicals tested on the basis of their superior performance as activating agents for Bacillus subtilis.

The optimal combination and concentrations of the five selected activating agents listed above were determined for each of the host organisms used in the studies reported below. The selected combinations were those found in preliminary studies to have the lowest possible concentrate that was capable of producing the desired protective effect. In this way, possible side effects and environmental pollution during the administration of these agents to the host organisms were avoided.

At the same time, the phytochemicals were screened for their ability to eliminate a cocktail of bacterial and fungal pathogens. For the purposes of comparison between the various treatments, fungal and bacterial elimination indices were calculated (0=maximal elimination, 5=no elimination).

The test mixtures, containing the glucose substrate and fungal and bacterial cocktails mentioned above together with all five of the activating phytochemicals and a penetrator (DMSO) and solvent (Triton) were used at four different concentrations: concentrations 1, 2, 3 and 4. In each case, the same amount of glucose substrate and fungal and bacterial cocktails—30 ml—was added to the mixture. Similarly, the concentration of the DMSO (0.5% v/v) and Triton (0.02% v/v) were the same in all mixtures. However, the concentrations of the Bacillus subtilis and each of the five activating agents (given in v/v %) were different in each of the test mixtures, as described in the Table I:

TABLE I

| | Con- | Con- | Con- | Con- |
|---|---|---|---|---|
| | centration 1 | centration 2 | centration 3 | centration 4 |
| Bacillus | 0.25% | 0.5% | 0.75% | 1.0% |
| subtilis | | | | |
| Sclareol | 0.1% | 0.2% | 0.3% | 0.4% |
| 98% | | | | |
| Naringin | 0.1% | 0.2% | 0.3% | 0.4% |
| 98% | | | | |
| Nootkatone | 0.1% | 0.2% | 0.3% | 0.4% |
| 98% | | | | |
| Stevia | 0.25% | 0.5% | 0.75% | 1.0% |
| CBD 3% | 0.1% | 0.2% | 0.3% | 0.4% |

Various different test mixtures containing different combinations of some or all of the five activating agents were used in this study, in accordance with the list of treatments given in Table II, below. In each case, the activating agents, Bacillus subtilis and substrate were used at the concentrations indicated in Table I. For example, when tested at Concentration 1, the concentration of sclareol in test mixtures containing that activating agent was 0.1%, while when tested at Concentration 2, sclareol was present at a concentration of 0.2%, and so on.

TABLE II

| | | Bacillus | | | | | |
|---|---|---|---|---|---|---|---|
| TEST | Substrate | subtilis | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | ✓ | — | — | — | — |
| 4 | ✓ | ✓ | — | ✓ | — | — | — |
| 5 | ✓ | ✓ | — | — | ✓ | — | — |
| 6 | ✓ | ✓ | — | — | — | ✓ | — |
| 7 | ✓ | ✓ | — | — | — | — | ✓ |
| 8 | ✓ | ✓ | ✓ | ✓ | — | — | — |
| 9 | ✓ | ✓ | ✓ | ✓ | ✓ | — | — |
| 10 | ✓ | ✓ | ✓ | ✓ | ✓ | ✓ | — |
| 11 | ✓ | ✓ | ✓ | ✓ | ✓ | ✓ | ✓ |
| 12 | ✓ | | ✓ | ✓ | ✓ | ✓ | ✓ |
| 13 | ✓ | | | ✓ | ✓ | ✓ | ✓ |
| 14 | ✓ | | | | ✓ | ✓ | ✓ |
| 15 | ✓ | | | | | ✓ | ✓ |
| 16 | ✓ | | | | | | ✓ |

### Results:

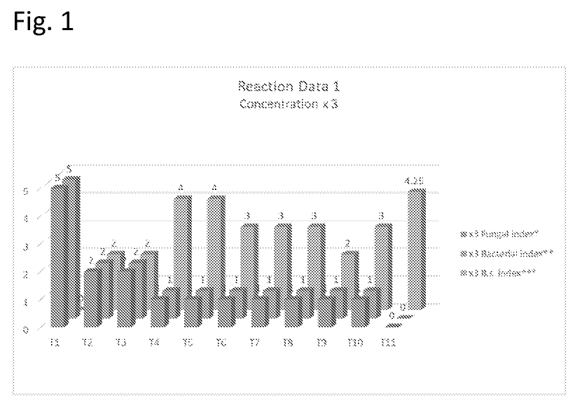

Preliminary results indicated that the optimal anti-fungal and anti-bacterial activity was obtained using test mixtures with concentration 2 and concentration 3 (see Table I, above). Since Bacillus subtilis colony development was optimal using concentration 3, this was the concentration that selected for use in the remainder of the study. The results obtained for fungal elimination, bacterial elimination and Bacillus subtilis activation (colony size) for the concentration 3 tests are summarized graphically in FIG. 1 in the front, middle and back rows of the graph, respectively. The eleven different treatments summarized in Table II, above, are labeled as T1 to T11 along the X axis of the graph.

As explained above, the three semi-quantitative indices used to assess the anti-fungal, anti-bacterial and activation properties are as follows:

Fungal index: 0 (no development) to 5 (maximum development)
Bacterial index: 0 (no development) to 5 (maximum development)
B.s index (colony forming index): 0 (no development) to 5 (maximum development)

It may be seen from FIG. 1 that the best results—both for Bacillus subtilis activation and for pathogen elimination were obtained using treatment 11, which (as shown in Table II, above) used a combination of all five activation agents.

The results of an additional experiment (in which 16 different agent mixtures were tested) are shown in the following table:

TABLE IIa

| | | Bacterial | Bacterial | Bacterial | Bacterial | Average |
|---|---|---|---|---|---|---|
| | | Index | Index | Index | Index | of 4 |
| Test No. | | rep 1 | rep 2 | rep 3 | rep 4 | reps: |
| 1 | Pathogen mix | 5 | 5 | 5 | 5 | 5.00 |
| 2 | 1 + 2 | 5 | 5 | 5 | 5 | 5.00 |
| 3 | 1 + 2 + 3 | 3 | 3 | 3 | 4 | 3.25 |
| 4 | 1 + 2 + 4 | 4 | 3 | 4 | 3 | 3.50 |
| 5 | 1 + 2 + 5 | 3 | 2 | 3 | 3 | 2.75 |
| 6 | 1 + 2 + 6 | 4 | 4 | 3 | 4 | 3.75 |
| 7 | 1 + 2 + 7 | 3 | 2 | 2 | 2 | 2.25 |
| 8 | 1 + 2 + 3 + 4 | 2 | 2 | 2 | 2 | 2.00 |
| 9 | 1 + 2 + 3 + 4 + 5 | 1 | 1 | 2 | 1 | 1.25 |
| 10 | 1 + 2 + 3 + 4 + 5 + 6 | 0 | 1 | 1 | 0 | 0.50 |
| 11 | 1 + 2 + 3 + 4 + 5 + 6 + 7 | 0 | 0 | 0 | 0 | 0.00 |
| 12 | 1 + 3 + 4 + 5 + 6 + 7 | 5 | 5 | 5 | 5 | 5.00 |
| 13 | 1 + 4 + 5 + 6 + 7 | 5 | 5 | 5 | 5 | 5.00 |
| 14 | 1 + 5 + 6 + 7 | 5 | 5 | 5 | 5 | 5.00 |
| 15 | 1 + 6 + 7 | 5 | 5 | 5 | 5 | 5.00 |
| 16 | 1 + 7 | 5 | 5 | 5 | 5 | 5.00 |

The results obtained with mixtures 12-16 are of particular interest: these mixtures do not contain B. subtilis, and their complete lack of activity against the pathogenic bacteria in this test system clearly indicate that the activating agents alone (including even a mixture of all five activating agents—test mixture 12 in Table IIa) are inactive. Thus, the presence of both the activating agents and B. subtilis (or another non-pathogenic bacterial species) are required in order to obtain the desired antimicrobial effect. It is to be further noted that this particular effect—the lack of activity of the activating agents alone, i.e. in the absence of B. subtilis or other non-pathogenic bacteria—was seen in all of the studies reported below (data not shown).

### Example 2

### Effect of Altering the Activating Agent Composition on the Activation of Bacillus subtilis and the Fungicidal and Bactericidal Activities of Said Composition

A second group of studies was aimed at investigating the effect of either eliminating one phytochemical from the full 5-component combination or of selectively altering the concentration of one or two components in the mixture.

### Material and Methods:

As for Example 1.

The various test mixtures were used at either concentration 3 or concentration 4 (as defined in Example 1, above). The composition of each of these test mixtures is summarized in the following two tables:

TABLE III
Concentration 3

| | | Bacillus | | | | | |
|---|---|---|---|---|---|---|---|
| TEST | Substrate | subtilis | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | — | ✓ | ✓ | — | ✓ |
| 4 | ✓ | ✓ | ✓ | ✓ | ✓ | — | ✓ |
| 5 | ✓ | ✓ | ✓ | ✓ | — | ✓ | ✓ |
| 6 | ✓ | ✓ | ✓ | — | ✓ | ✓ | ✓ |
| 7 | ✓ | ✓ | — | ✓ | ✓ | ✓ | ✓ |
| 8 | ✓ | ✓ | ✓ | ✓ | ✓* | ✓* | ✓ |

*In test 8, the Nootkatone and Stevia were each present at elevated concentration - 0.4% v/v Nootkatone (instead of 0.3%) and 1.0% Stevia (instead of 0.75%).

TABLE IV
Concentration 4

| | | Bacillus | | | | | |
|---|---|---|---|---|---|---|---|
| TEST | Substrate | subtilis | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | — | ✓ | ✓ | — | ✓ |
| 4 | ✓ | ✓ | ✓ | ✓ | ✓ | — | ✓ |
| 5 | ✓ | ✓ | ✓ | ✓ | — | ✓ | ✓ |
| 6 | ✓ | ✓ | ✓ | — | ✓ | ✓ | ✓ |
| 7 | ✓ | ✓ | — | ✓** | ✓ | ✓ | ✓ |

**In test 7, the Naringin was present at a reduced concentration - 0.3% v/v (instead of 0.4%).

### Results:

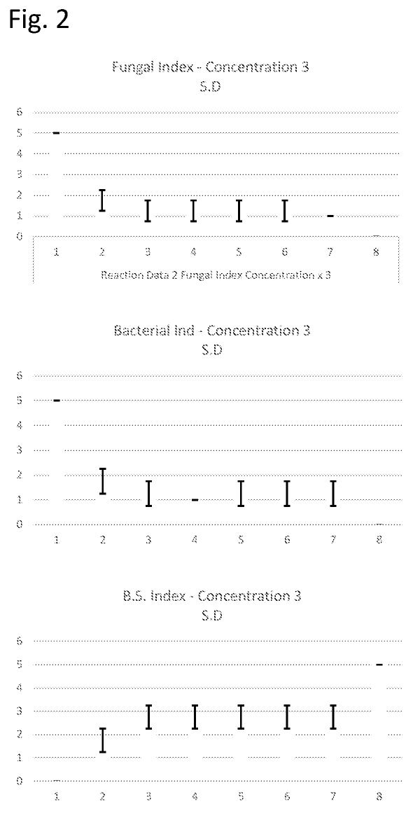

As may be seen in FIG. 2, all test mixtures containing 3 or 4 activating agents used at concentration 3 caused significant reduction in the fungal and bacterial indices (upper and middle graphs, respectively) and a significant increase in the Bacillus subtilis activation index (lower graph), when compared with medium only and medium plus Bacillus subtilis controls (mixtures 1 and 2, respectively).

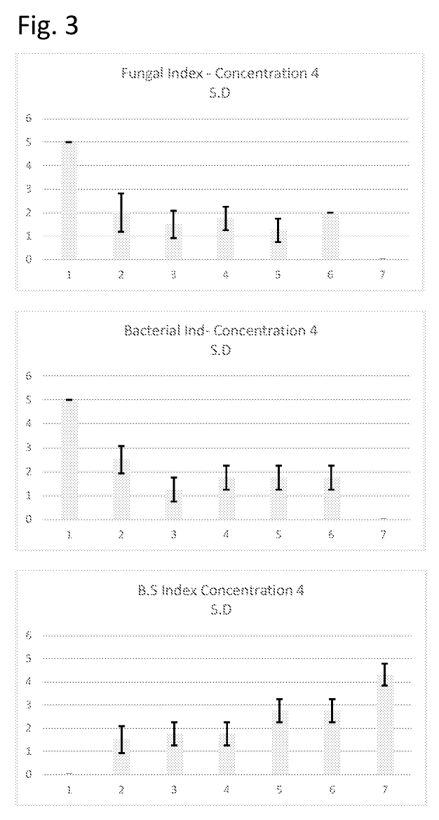

Similarly, as shown in FIG. 3, all test mixtures containing 3 or 4 activating agents used at concentration 4 caused significant reduction in the fungal and bacterial indices (upper and middle graphs, respectively) and a significant increase in the Bacillus subtilis activation index (lower graph), when compared with medium only and medium plus Bacillus subtilis controls (mixtures 1 and 2, respectively).

It may also be observed in FIG. 2 that the five-component activating agent mixture in which the Nootkatone and Stevia components are both at an elevated concentration (i.e. concentration 4, while all other components are at concentration 3; i.e. test mixture 8) has the greatest activity on all three indices.

Furthermore, FIG. 3 shows that the four-component activating agent mixture (number 7) in which the naringin concentration is reduced to concentration 3, with all other components at concentration 4, has the greatest activity in this data set, as measured by all three indices.

These data indicate that mixtures containing less than the maximum five activating agents may be used to protect host organisms from fungal or bacterial attack. In addition, these results also indicate that optimization of the mixtures may be obtained by manipulating the concentration of one or more individual activating agents in the mixture.

### Example 3

### The Fungicidal and Bactericidal Activities of Various Activating Agent Compositions in Conjunction with a Different Bacillus subtilis Formulation

In this study, the experiments performed in Example 2, above, were repeated using a different B. subtilis preparation.

### Material and Methods:

As for Example 1.

The various test mixtures were used at either concentration 3 or concentration 4 (as defined in Example 1, above). The composition of each of these test mixtures is as summarized in Tables III and IV in Example 2, hereinabove.

### Results:

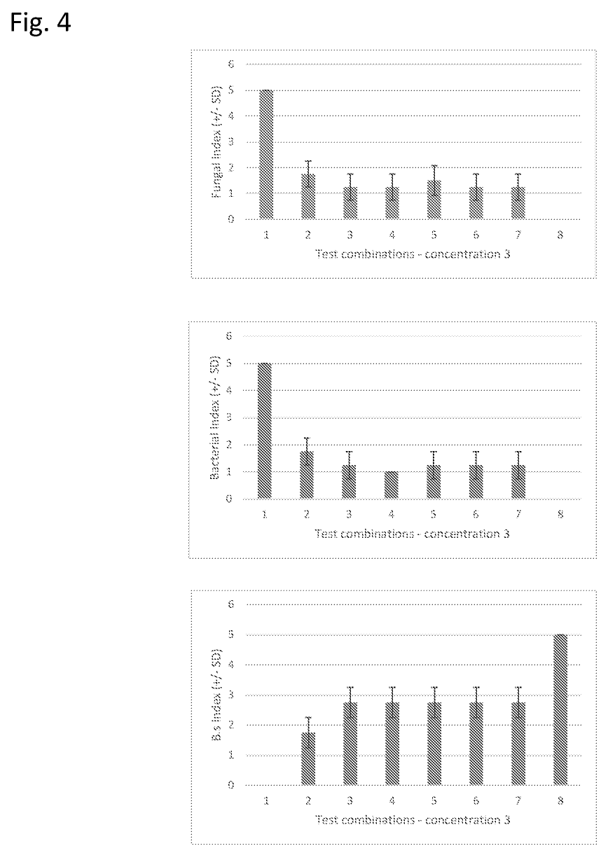

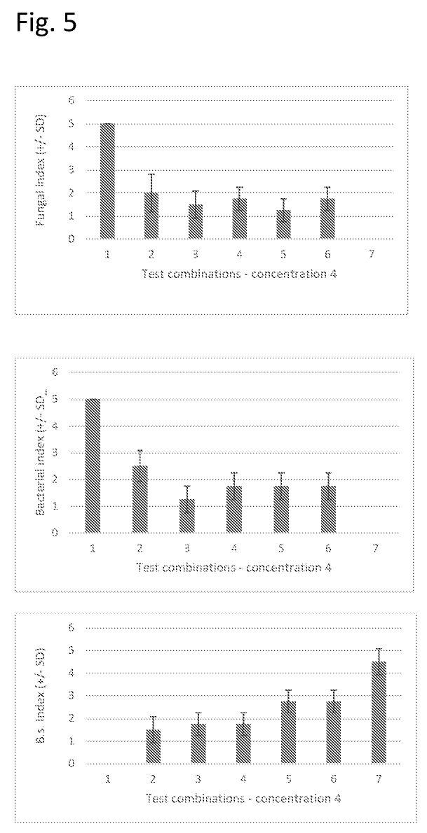

This study confirms the results obtained in Example 2. Thus, as seen in FIG. 4 (concentration 3) and FIG. 5 (concentration 4), all test mixtures containing 3, 4 or 5 activating agents at concentration 3, caused a marked reduction in the fungal and bacterial indices (upper and middle graphs, respectively). Furthermore, they also caused a significant increase in the Bacillus subtilis activation index (lower graph).

Of particular note is the fact that (as in the case of the study reported in Example 2 using the Serenade® preparation), at concentration 3, the five-component activating agent mixture in which the Nootkatone and Stevia components are both at an elevated concentration (i.e. concentration 4, while all other components are at concentration 3; i.e. test mixture 8) has the greatest activity on all three indices (FIG. 4). Similarly, as shown in FIG. 5, the four-component activating agent mixture (number 7) in which the naringin concentration is reduced to concentration 3, with all other components at concentration 4, has the greatest activity in this data set, as measured by all three indices.

These results, obtained with the Cease® Bacillus subtilis formulation confirm the findings obtained with the Serenade® formulation (Example 2, hereinabove), indicating that the effects observed are not specific to any one particular Bacillus subtilis preparation.

### Example 4

### Anti-Inflammatory Activity of Agents Used in the Present Invention

Following the results obtained with combinations of Bacillus subtilis and some or all of the five activating agents reported in Examples 1-3, hereinabove, said agents were investigated in order to look for common functional properties, in addition to their bactericidal, fungicidal and B. subtilis-activating abilities.

Following a series of preliminary investigations, the present inventors unexpectedly found that each of the five activating agents tested in the studies presented hereinabove, also share a highly potent anti-inflammatory activity.

In order to investigate this further, three of the activating agents used in the previous Examples—both separately, in combination with each other and in combination with Bacillus subtilis, were tested for their ability to inhibit the in vitro production in a cultured macrophage line of two key inflammatory inhibitors: nitric oxide (NO) and TNF-α. In addition, the viability of the macrophages was measured at appropriate IC₅₀ values corresponding to the inhibition of NO and TNF-α, at the time that the anti-inflammatory assays were performed.

### Methods:

### RAW 264.7 Macrophage Cell Line:

RAW 264.7 macrophages were grown in flat-bottomed flasks using a standard growth medium (DMEM supplemented with 5% FBS, antibiotics and glutamine. The cells were maintained in accordance with standard procedures well known in the art. After the cells reached confluence, they were removed from the flasks using mechanical means and then concentrated by centrifuging and resuspended in a small volume of fresh culture medium. The cell concentration was adjusted with growth medium in order that about 75,000 cells could be added to each well of a 96-well plate. A combination of 25 g/mL LPS and 10 U/ml IFN-γ DMEM, was used for activation of the macrophages. The various test agents were added to the wells one hour prior to activation. The cells were then incubated for a further 24 hours, prior to assaying the inflammatory mediator production and cell viability.

### Determination of Cell Viability:

The Alamar Blue assay of viability was performed by adding 100 μL of a 10% Alamar Blue solution to each well and incubating at 37° C. for 1-2 hr. Fluorescence was measured (excitation at 545 nm and emission at 595 nm) and expressed as a percentage of the values in untreated control cells.

### Determination of Nitric Oxide Production by Griess Assay:

The production of NO by the macrophages subjected to the various treatments was assayed using the Griess reagent (equal volumes of 1% sulphanilamide and 0.1% napthyethylene-diamine in 5% HCl). 70 μL of supernatant from each test well was transferred to a fresh 96-well plate and mixed with 70 μL of Griess reagent and the violet color produced was measured at 540 nm.

### TNF-α Determination by ELISA:

A sandwich ELISA was used to determine TNF-α concentration. The primary antibody was used at a concentration of 0.5 g/mL in PBS. Serial dilutions of TNF-α standard from 0 to 1000 pg/mL in diluent (0.05% Tween-20, 0.1% BSA in PBS) were used as internal standard. TNF-α was detected with a biotinylated second antibody and an avidin peroxidase conjugate with TMB as detection reagent. The color development was monitored at 655 nm, taking readings after every 5 minutes. After 25 minutes, the reaction was stopped using 0.5 M sulphuric acid and the absorbance was measured at 450 nm.

### Tested Agents:

The methods described above were used to determine the effects of Sclareol, Naringin and Steviol, and their combinations with each other and with Bacillus subtilis, on NO and TNF-α production, and on cell viability. The results for the anti-inflammatory activities are presented as IC₅₀ values for the inhibition of NO and TNF-α production in Table V, below, together with the cell viability results. In addition, comparable results obtained from the scientific literature (A. S. Ravipati et al. (2012) BMC Complementary and Alternative Medicine, 12:173 “Antioxidant and anti-inflammatory activities of selected Chinese medicinal plants and their relation with antioxidant content”) for two additional plant species—aqueous extracts of Aster tataricus and Cyperus rotundus—are presented at the end of the table. Extracts of these two species were investigated by the present inventors with regard to their fungicidal and bactericidal effects in combination with B. subtilis. The results of these investigations are presented in Example 5, hereinbelow.

### Results:

The results obtained for the anti-inflammatory and viability assays of the cultured macrophages treated with the various agents are presented in Table V, below.

TABLE V

| | IC₅₀ for the | | IC₅₀ for the | |
|---|---|---|---|---|
| | inhibition | Cell | inhibition | Cell |
| Name of agent/ | of NO | viability | of TNF-α | viability |
| combination/plant | production | (% of cell | production | (% of cell |
| extract tested | (mg/ml) | survival) | (mg/ml) | survival) |
| 1. Sclareol | 0.04 ± 0.02 | 96.20 ± 2.1 | 0.08 ± 0.02 | 95.30 ± 2.2 |
| Sclareol + B.s. | 0.02 ± 0.01 | 97.30 ± 3.2 | 0.04 ± 0.01 | 94.40 ± 4.1 |
| 2. Naringin | 0.04 ± 0.02 | 90.10 ± 4.2 | 0.09 ± 0.02 | 91.50 ± 5.2 |
| Naringin + B.s. | 0.03 ± 0.01 | 92.60 ± 3.0 | 0.03 ± 0.01 | 93.20 ± 3.2 |
| 3. Steviol | 0.06 ± 0.03 | 98.20 ± 4.2 | 0.08 ± 0.02 | 99.54 ± 4.1 |
| Steviol + B.s. | 0.03 ± 0.01 | 96.43 ± 5.3 | 0.03 ± 0.004 | 95.45 ± 5.2 |
| 1 + 2 + 3 | 0.004 ± 0.002 | 95.76 ± 4.5 | 0.01 ± 0.003 | 92.36 ± 2.1 |
| 1 + 2 + 3 + B.s. | 0.001 ± 0.001 | 98.40 ± 3.3 | 0.003 ± 0.002 | 96.46 ± 4.1 |
| Bacillus subtilis | NA | 89.5 ± 3.5 | NA | 86.55 ± 2.0 |
| Aster tataricus | 0.14 ± 0.08 | 98.95 ± 1.5 | 2.30 ± 0.09 | 99.70 ± 0.5 |
| Aster tataricus + | 0.09 ± 0.01 | 97.44 ± 1.1 | 1.80 ± 0.03 | 9.65 ± 0.2 |
| B. subtilis | | | | |
| Cyperus rotundus | 0.35 ± 0.37 | 86.60 ± 19 | 2.39 ± 0.64 | 107.50 ± 10.6 |
| Cyperus rotundus + | 0.21 ± 0.16 | 86.50 ± 11 | 1.22 ± 0.13 | 96.80 ± 2.1 |
| B. subtilis | | | | |
| Platycodon grandiflorus | 0.43 ± 0.08 | 83.05 ± 3.6 | 1.79 ± 0.11 | 59.45 ± 2.1 |
| Platycodon grandiflorus + | 0.21 ± 0.04 | 84.06 ± 0.21 | 1.12 ± 0.13 | 60.11 ± 2.2 |
| B. subtilis | | | | |
| Pleione bulbocadioides | 0.58 ± 0.39 | 101.5 ± 0.7 | 1.59 ± 0.22 | 98.6 ± 2.0 |
| Pleione bulbocadioides + | 0.22 ± 0.15 | 99.80 ± 0.3 | 1.23 ± 0.16 | 98.5 ± 1.5 |
| B. subtilis | | | | |

It may be seen that none of the treatment agents tested had any significant adverse effect on the viability of the macrophages. Consequently, any inhibition of the production of the two inflammatory mediators caused by these agents was not a result of a general cytotoxic effect.

It is to be noted from the table that when taken separately, the IC₅₀ for NO inhibition of the three agents Sclareol, Naringin and Steviol are 0.04, 0.04 and 0.02, respectively. Furthermore, when used in combination with each other, said combination is even more potent, with an IC₅₀ for NO inhibition of 0.004 in the absence of B. subtilis, and 0.001 in the presence of B. subtilis. If these results are compared with the comparable IC₅₀ values for NO inhibition published for 44 selected plant extracts in the aforementioned paper by A. S. Ravipati et al. (2012), it will be seen that the values for Sclareol, Naringin and Steviol are at the lower extremity of the range of values in said paper (0.03-1.49), and in one case (Steviol) even beyond the lowest extent of that range. Similarly, if the mean value for Sclareol, Naringin and Steviol is compared with that for the 44 plants reported in the paper, it may be noted that the former (0.03) is much lower than the mean extracted from said published values (0.26).

A similar conclusion may also be drawn with regard to the inhibition of TNF-α by Sclareol, Naringin and Steviol when tested separately, with IC₅₀ values of 0.08, 0.09 and 0.08, respectively (range=0.08-0.09; mean=0.083), compared with the published results for the 44 plant extracts in A. S. Ravipati et al. (2012) (range=0.07-2.5; mean—1.04).

It may thus be concluded that the three agents selected and tested in Examples 1-3, hereinabove, all have anti-inflammatory activity, and are more potent (i.e. have a lower IC₅₀) than most of a set of 44 herbal extracts, commonly used in Chinese medicine (A. S. Ravipati et al. (2012)), with respect to NO and TNF-α inhibition.

Furthermore, it is of interest to note from Table V that even in the case of less potent anti-inflammatory plant extracts (e.g. Aster tataricus, Cyperus rotundus, Platycodon grandiflorus and Pleione bulbocadioides), said extracts are also effective as activating agents for Bacillus subtilis with regard to anti-fungal and anti-bacterial activity (as will be shown in Example 5, hereinbelow).

### Example 5

### The Fungicidal and Bactericidal Activities of Combinations of Two Different Plant Extracts with Bacillus subtilis

The potential for extracts of two plants with anti-inflammatory properties—Aster tataricus and Cyperus rotundus—to have a potentiating effect on the fungicidal and bactericidal activities of Bacillus subtilis was investigated.

### Method:

The same methods for assaying anti-fungal and anti-bacterial properties as used in Example 1, hereinabove, were applied in this study to combinations of Bacillus subtilis with aqueous extracts of Aster tataricus and Cyperus rotundus. Two different concentrations of the extracts and of the B. subtilis suspension were used, as summarized in Table VI, below.

TABLE VI

| | Concentration 3 | Concentration 4 |
|---|---|---|
| Bacillus subtilis | 0.75% | 1.0% |
| Aster tataricus | 0.9% | 1.2% |
| Cyperus rotundus | 0.9% | 1.2% |

Various combinations of the extracts and the B. subtilis suspension were prepared and tested in accordance with the scheme provided in Table VII. All of these combinations were tested both at concentration 3 and at concentration 4.

TABLE VII

| | | Bacillus | Aster | Cyperus |
|---|---|---|---|---|
| TEST | Substrate | subtilis | tataricus | rotundus |
| 1 | ✓ | — | — | — |
| 2 | ✓ | ✓ | — | — |
| 3 | ✓ | ✓ | ✓ | — |
| 4 | ✓ | — | ✓ | — |
| 5 | ✓ | ✓ | — | ✓ |
| 6 | ✓ | — | — | ✓ |

### Results:

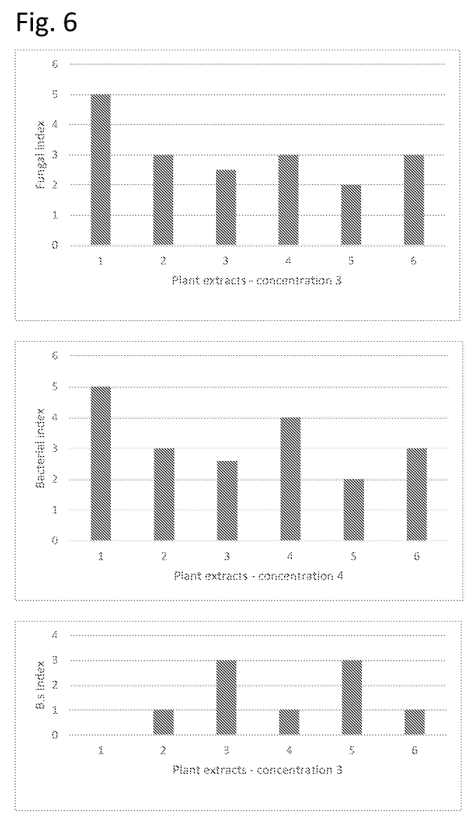

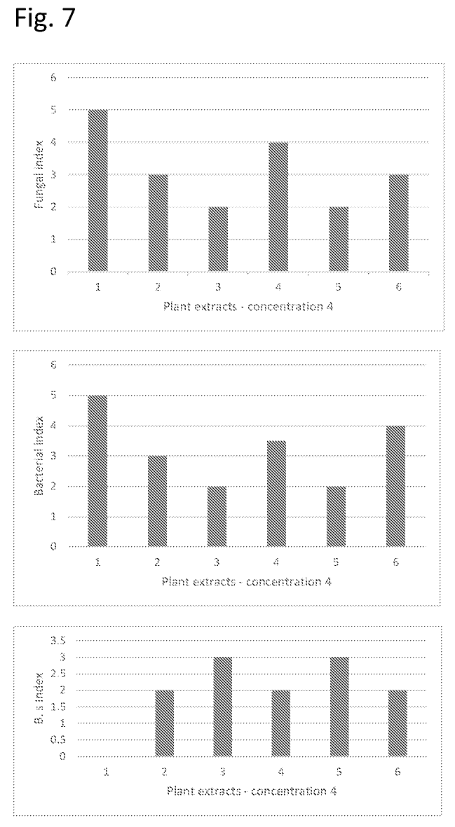

FIG. 6 graphically presents the results obtained using the combinations of Table VII at concentration 3. It will be seen from the upper graph that all of the combinations tested displayed fungicidal activity, and that the most effective combination in this regard was combination 5, namely B. subtilis together with the extract of Cyperus rotundus. Similar results are obtained at concentration 4, as shown in the upper graph in FIG. 7. In this case, however, the combination of WB. subtilis and Aster tataricus also produced a similar result. With regard to bactericidal activity, the middle graph of FIG. 6 indicates that at concentration 3, the combination of B. subtilis and Cyperus rotundus causes the largest reduction in the number of bacterial cells of all of the combinations tested. With regard to the concentration 4 data, however, the results of the bactericidal study shown in the middle graph of FIG. 7 show that—as in the cases of the concentration 4 fungicidal data discussed above—both binary combinations of plant extract with B. subtilis produced the largest bactericidal effect.

Finally, with regard to activation of B. subtilis, it is clear from the lower graphs of both FIG. 6 and FIG. 7 that each of the binary combinations of plant extract with B. subtilis show the maximum effect.

It may be concluded from this preliminary study that aqueous extracts of two plants having anti-inflammatory properties act as activating agents for Bacillus subtilis, increasing its anti-bacterial and anti-fungal activities.

### Example 6

### Inoculation of Cucumber Seedlings

### Method:

10 cc of each mixture of activating agents, Bacillus subtilis and additional components (including the bacterial and/or fungal cocktails as described in Tables I and II of Example 1, hereinabove) was sampled from the relevant petri dish and injected into 4 replicates of germinating cucumber seeds 10 hour after sowing.

The health of each plant was assessed 5 days following treatment, using a semi-quantitative inoculation index (0=healthy, 5=dead).

### Results

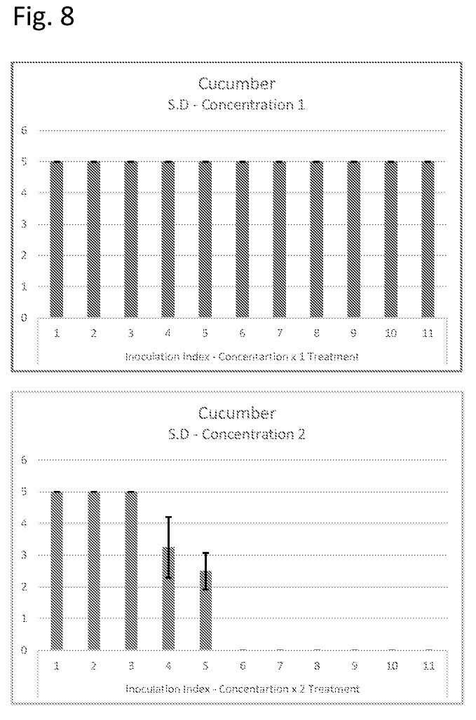

The results of this study are shown graphically in FIG. 8, in which the four separate graphs summarize the data obtained using the activating agents at concentrations 1, 2, 3 and 4 (from above to below).

As may be seen from the first (upper) graph in FIG. 8, none of the treatment protocols, when used at the lowest concentration (concentration 1) was capable of protecting the plants from microbial infection (inoculation index of 5 for all treatments).

The second graph in FIG. 8 indicates that at the next higher concentration in the series (concentration 2), activation agent mixtures 6 to 11 all provide full protection for the cucumber plants from fungal and bacterial infection. A similar result was also seen when the agents were used at concentration 3, as shown in the third graph in FIG. 8.

At the highest concentration (concentration 4; last graph in FIG. 8), the protective effect is seen with activation mixtures 5 to 11.

In summary: all of the multiple-component activating agent mixtures, as well as some of the mixtures containing only one activating agent, were effective at protecting cucumber plants in vivo, when used at concentrations 2 to 4. The semi-quantitative data obtained in this study correlate very well with the appearance of the plants that were subjected to the various treatments.

### Example 7

### Inoculation of Tomato Seedlings

### Method:

Tomato seedlings were inoculated with test mixtures containing Bacillus subtilis and various combinations of activating phytochemicals, in the same manner as in Example 6, hereinabove. However, the composition and concentration of the various test mixtures used were different from that of Example 6, and are summarized in the following two tables (all concentrations are given as % v/v):

TABLE VIII

| | Concentration | Concentration | Concentration |
|---|---|---|---|
| | 2 | 3 | 4 |
| Bacillus subtilis | 0.5% | 0.75% | 1.0% |
| Sclareol 98% | 0.2% | 0.3% | 0.4% |
| Naringin 98% | 0.2% | 0.3% | 0.4% |
| Nootkatone 98% | 0.2% | 0.3% | 0.4% |
| Stevia | 0.5% | 0.75% | 1.0% |
| CBD 3% | 0.2% | 0.3% | 0.4% |

In addition, the mixtures all contained a penetrator (DMSO) at a concentration of 0.5% v/v and a solvent (Triton) at a concentration of 0.02% v/v.

TABLE IX

| | | Bacillus | | | | | |
|---|---|---|---|---|---|---|---|
| TEST | Substrate | subtilis | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | ✓ | ✓ | — | — | — |
| 4 | ✓ | ✓ | ✓ | ✓ | ✓ | — | — |
| 5 | ✓ | ✓ | ✓ | ✓ | ✓ | ✓ | — |
| 6 | ✓ | ✓ | ✓ | ✓ | ✓ | ✓ | ✓ |
| 7*** | ✓ | ✓ | ✓/— | ✓ | ✓ | ✓ | ✓ |

***Test mixture 7 was omitted in the sets performed at concentration 2. At concentration 3, this mixture contained Nookatone and Stevia at an elevated concentration (0.4% and 1.0%, instead of 0.3% and 0.75%, respectively). At concentration 4, Sclaerol was omitted and Naringin was used at a lower concentration (0.3% instead of 0.4%).

### Results

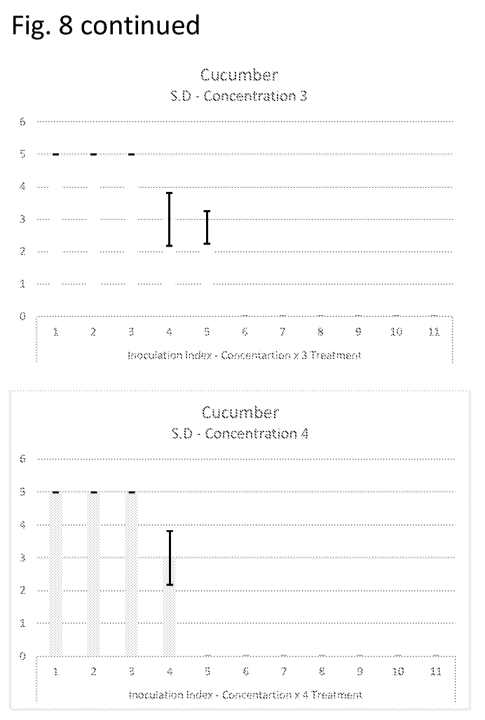

The results of this inoculation study are summarized graphically in FIG. 9. It may be seen from this figure that at concentration 2 (upper graph), only test mixture 6 caused near-maximal protection of the tomato plants. At concentrations 3 and 4 (middle and lower graphs, respectively), however, both treatments 6 and 7 resulted in maximal protection.

### Example 8

### Tomato Plants—Field Study

### Introduction:

The main growing region for tomatoes in Israel is the southwest of the country.

Two years ago, the area was heavily infected by a new aggressive strain of Tomato mosaic virus (ToMV). This virus infected the plants and the commercial yield was reduced to less than half of the normal yield level. Unfortunately, no genetic resistant varieties yet exist.

A field study using the presently-disclosed method was performed in order to assess whether said method could increase the survival of ToMV-infected tomato plants.

### Method:

A trial of bacillus subtilis and activating phytochemicals was performed in a tomato net-house that was heavily infected by the virus in the previous growing season.

The treatment mixtures used were the same as those described hereinabove in Example 1, used at concentration 2.

The implementation methods used were:

### a) Spraying;

b) Spraying and fertigation;

### c) Fertigation;

d) Untreated control.

The tomato plants were located in either south-facing or north-facing plots, and each were treated 6, 5 and 4 times every 14 days.

### Results

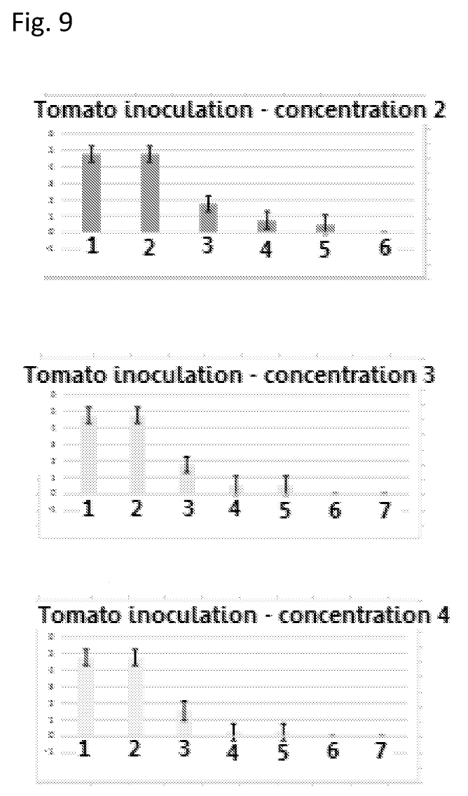

The results of this study are presented graphically in FIG. 10. The first three graphs in this figure relate to the plants grown in the North-facing plots, while the second set of three graphs present the results for the plants grown in the South-facing plots.

As may be seen from this figure, the 6-treatment and 5-treatment regimens (first and second graphs, from above) both yielded good results (as evidenced by a reduced inoculation index) when the composition was administered either by fertigation (bars 5 and 6 in each set) or by a combination of fertigation and spraying (bars 3 and 4).

It also appears from these results that the tomato plants in the South-facing rows responding more to these treatments and implementation methods than those in the North-facing plots.

### Example 9

### Pepper Plant Seedlings

### Method:

The same method for inoculating tomato seedlings, as described hereinabove in Example 7, was employed to test the activity of the composition of the present invention on pepper plant seedlings. The treatment protocol was also the same as used in Example 7, as set out in Tables V and VI, hereinabove.

### Results:

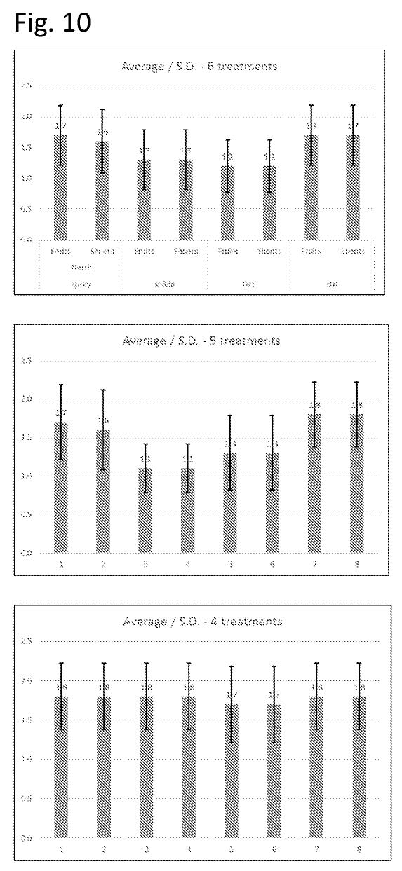

The results for the treatment of the pepper plant seedlings are summarized in FIG. 11. The upper graph presents the results for the activation mixtures used at concentration 2, the middle graph relates to mixtures used at concentration 3, and the lower graph presents the results for the concentration 4 mixtures.

It may be seen from these graphs that at concentration 2, activation mixtures 5 and 6 provided complete protection for the seedlings. At concentrations 3 and 4, however, activation mixture 4 provided nearly complete protection, in addition to the complete protection provided by activation mixtures 5 and 6.

### Example 10

### Maize Seedling Inoculation

### Method:

The same method for inoculating the tomato and pepper seedlings, as described hereinabove in Examples 7 and 9, was used to inoculate maize (Zea mays ssp. Mays) with the activation agent mixtures (at four different concentrations) defined in Tables I and II of Example 1. The only difference between the treatment protocol used in this study and that described in the previous examples relates to the composition of the bacterial and fungal cocktails used. Thus, in this study, the fungal cocktail comprised Pythium spp., Rizoctonia spp. and Penicilium oxalicum, while the bacterial cocktail contained Erwinia chrysanthemi, Erwinia dissolvens and Enterobacter dissolvens.

### Results:

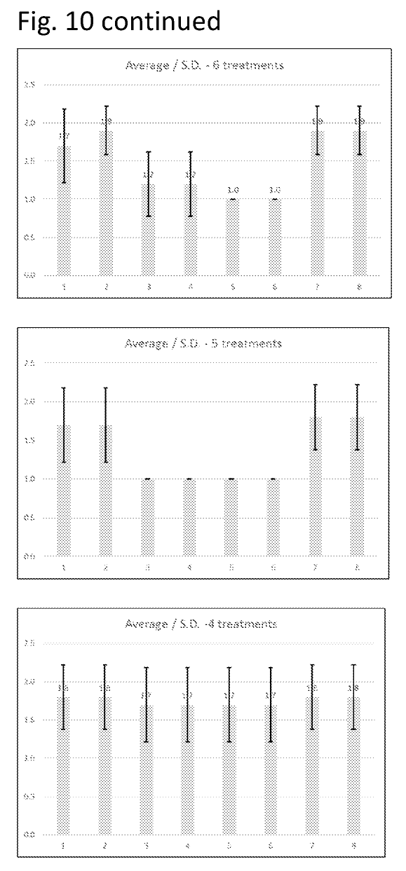

The results of this inoculation study are graphically summarized in FIG. 12, with the four graphs presenting the results for the eleven activation mixtures used at concentration 1, 2, 3 and 4 (in descending order).

It may be seen from these graphs that the best protection against fungal and bacterial infection was provided by activation mixture 11, when used at concentrations 2, 3 and 4.

### Example 11

### Wheat Seedlings

### Method:

The same method for inoculating the tomato and pepper seedlings, as described hereinabove in Examples 7 and 9, was used to inoculate wheat seedlings (Triticum aestivum) with the activation agent mixtures (at four different concentrations) defined in Tables I and II of Example 1. The only difference between the treatment protocol used in this study and that described in the previous examples relates to the composition of the bacterial and fungal cocktails used. Thus, in this study, the fungal cocktail comprised Rizoctonia solani, Pythium graminicola, and Pythium myriotylum, while the bacterial cocktail contained Pseudomonas syrigae, Xanthomonas campestris, and Erwinia rhapontici.

### Results:

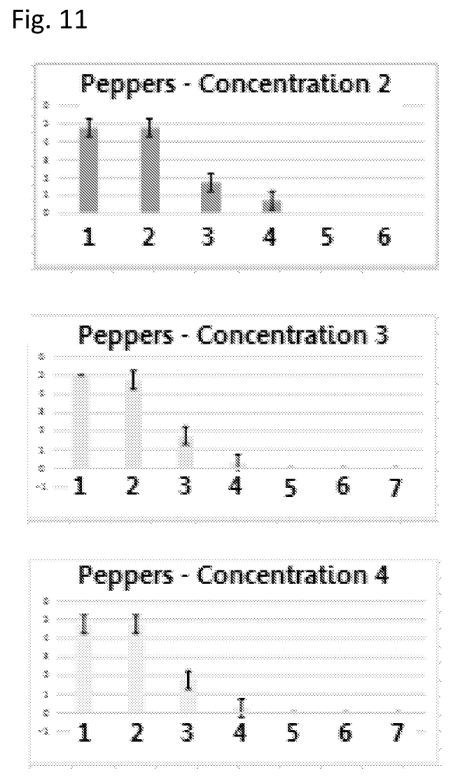

The results of this inoculation study are graphically summarized in FIG. 13, with the four graphs presenting the results for the eleven activation mixtures used at concentration 1, 2, 3 and 4 (in descending order).

It may be seen from these graphs that the best protection against fungal and bacterial infection was provided by activation mixture 11, when used at concentrations 2, 3 and 4 and activation mixture 10, when used at concentrations 3 and 4.

### Example 12

### Rice Seedlings

### Method:

The same method for inoculating the tomato and pepper seedlings, as described hereinabove in Examples 7 and 9, was used to inoculate rice seedlings (Oryza sativa) with the activation agent mixtures (at four different concentrations) defined in Tables I and II of Example 1. The only difference between the treatment protocol used in this study and that described in the previous examples relates to the composition of the bacterial and fungal cocktails used. Thus, in this study, the fungal cocktail comprised Pythium spinosum, Rizoctonia solani, Pythium dissotocum, while the bacterial cocktail contained Xanthomonas campestris pv. Oryzae and Erwinia chrisantemi.

### Results:

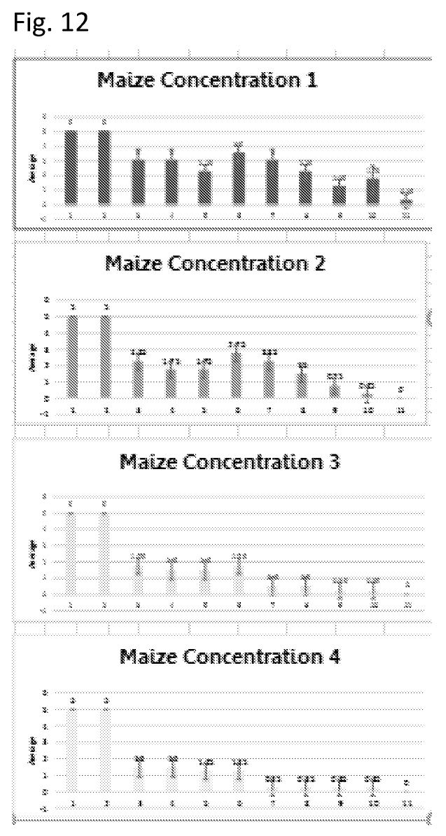

The results of this inoculation study are graphically summarized in FIG. 14, with the four graphs presenting the results for the eleven activation mixtures used at concentration 1, 2, 3 and 4 (in descending order).

It may be seen from these graphs that the best protection against fungal and bacterial infection was provided by activation mixture 11, at all four concentrations tested, mixture 10 when used at concentrations 3 and 4, mixture 9 at concentrations 3 and 4 and activation mixture 8, when used at concentration 4 only.

### Example 13

### Chickpeas—Field Study

### Method:

The field trial was sown on Oct. 9, 2015 in heavy soil in Nahalal, Israel.

The normal sowing period for chickpeas (Cicer arietinum L) in Israel is at the beginning of February, mainly because of two main pathogens—Ascochyta rabiei and Fusarium oxysporum f. sp. cicero.

The existing varieties have mild tolerance to these pathogens and cannot cross the Israeli winter.

The field chosen for this trial had a high Fusarium loading in the past.

Prior to Feb. 16, 2016, the field was not treated and heavy symptoms of both of 2 pathogens were observed all over the field.

On Feb. 16, 2016, the first treatment was applied.

4 chickpea varieties were included in the trial—1, 11, 12, and 13—and plants of each variety were subjected to one of three different protocols: two different treatment protocols (red and blue protocols; see Table X, below) and a non-treated control protocol:

TABLE X

| | Red protocol | Blue protocol |
|---|---|---|
| Material | (single concentration) | (double concentration) |
| Bacillus subtilis | 0.5% | 0.5% |
| Sclareol | 0.1% | 0.2% |
| Naringin | 0.1% | 0.2% |
| Nootkatone | 0.1% | 0.2% |
| Steviol | 0.1% | 0.2% |
| CBD | 0.1% | 0.2% |
| DMSO | 0.5% | 0.5% |
| Triton | 0.025% | 0.025% |

The following different application procedures were used to treat the plants with the treatment and control protocols:
1. 1 spray on the Feb. 16, 2016.
2. 1 fertigation on Feb. 16, 2016.
3. 1 spray+fertigation on Feb. 16, 2016.
4. 2 sprays+fertigation on Feb. 16, 2016 and on Mar. 1, 2016.

The success or otherwise of the various treatments was determined by measuring the yield of each treatment area, and then extrapolating that weight to Kg per Dunam (1000 m²).

### Results

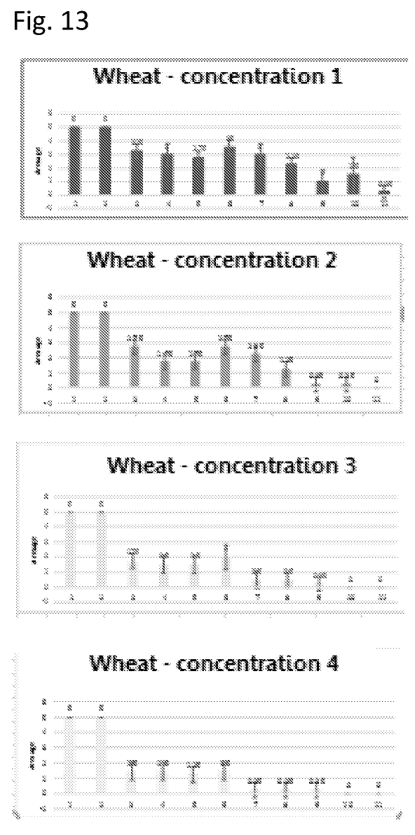

The results of the various treatments are summarized graphically in FIG. 15. It will be noted from this figure that chickpea variety 13 responded to both activation mixture treatments (blue and red protocols) to a much greater extent than the other varieties tested, regardless of the manner in which the treatment was administered Thus, the extrapolated yield of variety 13 with the blue protocol and the best application route is 684 kg.

It is to be noted that this yield is 4.2 times greater than the control and more than double of the best commercial yield level expected in Israel.

The treatment with the blue protocol made it possible to do the sowing 4 months earlier and obtain these yield results.

It is to be noted that the frequency of the treatments with the protocols was checked and it was becoming clear that the best results were obtained when the treatment frequency was very intensive. This suggests that it may be advantageous to administer the compositions of the present invention via a slow and/or controlled release membrane exposing the host species to constant levels of said composition. This possibility was further investigated, and the results presented hereinbelow (Example 18).

### Example 14

### In Vitro Study of Bacterial Pathogens of Relevance for Dental Caries in Human Subjects

### Introduction:

In this preliminary study, the effects of the composition of the present invention were tested in vitro on two bacterial species that have been implicated in the development of dental caries in human subject:
1. Streptococcus sobrinus.
2. Lactobacilli.

When resident in the oral cavity, these bacteria derive their nutrition primarily from ingested sucrose. Consequently, for the purpose of the present in vitro study, sucrose was also used as the growth substrate.

### Method:

For the purposes of comparison between the various treatments, an inoculation index was calculated (0=maximal elimination, 5=no elimination).

The test mixtures, containing 30 ml of bacteria being tested suspended in 50% v/v sucrose, together with all five of the activating phytochemicals and a penetrator (DMSO) and solvent (Triton) were used at four different concentrations: concentrations 1, 2, 3 and 4. In each case, the same amount of bacterial suspension—30 ml—was added to the mixture. Similarly, the concentration of the DMSO (0.5% v/v) and Triton (0.02% v/v) were the same in all mixtures. However, the concentrations of the Bacillus subtilis and each of the five activating agents (given in v/v %) were different in each of the test mixtures, as described in Table I, in Example 1, hereinabove.

Various test mixtures containing different combinations of some or all of the five activating agents were used in this study, in accordance with the list of treatments given in Table XI, below.

TABLE XI

| | | Bacillus | | | | | |
|---|---|---|---|---|---|---|---|
| TEST | Substrate | subtilis | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | ✓ | ✓ | — | — | — |
| 4 | ✓ | ✓ | ✓ | ✓ | ✓ | — | — |
| 5 | ✓ | ✓ | ✓ | ✓ | ✓ | ✓ | — |
| 6 | ✓ | ✓ | ✓ | ✓ | ✓ | ✓ | ✓ |

### Results:

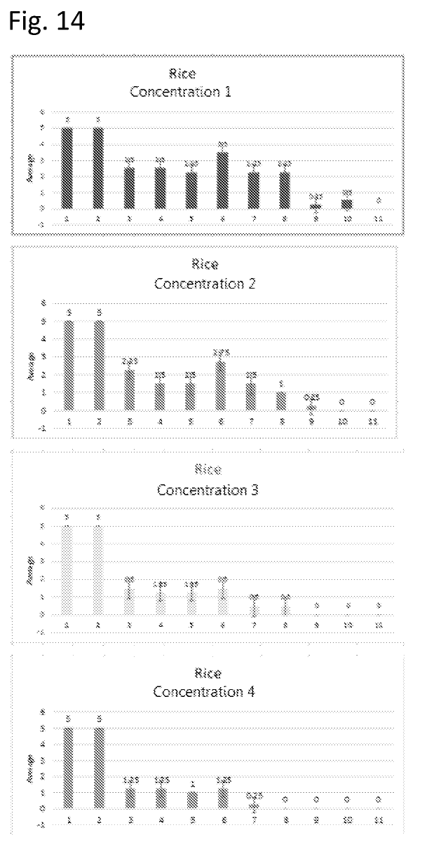

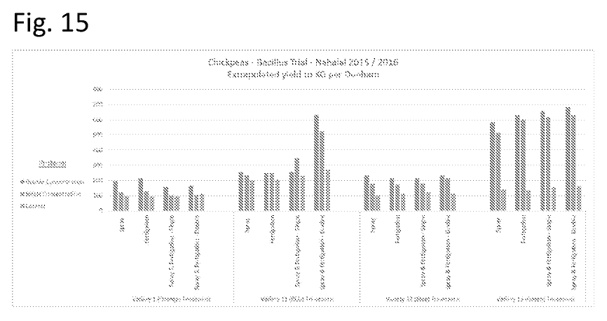

The results obtained with the test bacteria Streptococcus sobrinus are shown in FIG. 16, while the corresponding results obtained with Lactobacilli are presented in FIG. 17.

Almost complete elimination of the bacteria (in the case of both Streptococcus sobrinus and Lactobacilli) is seen when activation mixture 6 is used—regardless of the concentration used. In addition, almost maximal elimination is observed when mixture 5 is used at concentrations 2, 3 or 4. This latter result would suggest that the phytochemical not present in mixture 5—CBD—may not be essential to obtain the observed protective effect when challenged with either of the two cariogenic bacterial species tested in this study. It is possible, however, that mixture 6, containing all five phytochemicals (i.e. including CBD) will assure more consistent results.

### Example 15

### Preliminary Field Trial Using Compositions of the Present Invention for the Protection of Carrots Against Infection with Candidatus Liberibacter

### Introduction:

Candidatus Liberibacter is a genus of Gram-negative bacteria in the Rhizobiaceae family. It has not so far been possible to maintain these bacteria in culture, and their detection and quantification is generally accomplished using PCR amplification of their 16S rRNA gene with specific primers. Members of the genus are plant pathogens mostly transmitted by psyllids.

These bacteria can infect carrot and citruses (greening disease), and in so doing, may cause significant commercial damage.

The aim of this study was to investigate whether compositions of the present invention comprising a combination of B. subtilis and activating agents is capable of protecting carrot plants during the entire growing season.

### Materials and Methods:

Perlite granules were soaked in preparations containing combinations of B. subtilis and activating agents, and then coated with a slow-release polymer.

The granules were placed underneath the sowing trench, and following sowing, the plants and carrots were monitored for the development of symptoms of infections with Candidatus liberibacter.

The details of the trial protocol are as follows:

1. The sowing was done on the 8 Feb. 2017-3 triple rows per bed of 193 cm.
2. The trial area was covered with Agrinet netting until complete germination.
3. Irrigation was performed with mini-sprinklers providing 4 qm/d.
a) During germination 70% penman every day.
b) After germination 90% penman every 4 days.
   4. Fertilizers: During months 2 and 3 of the trial: 10 units of nitrogen.
   5. Fungicides: Every 10 days, treatment against Oidium and Oidiopsis mildews using Polar (Amiran K Ltd., Nairobi, Kenya), Shavit (Adama Ltd., Israel), and Ami-oz.)

### 6. Herbicides:

a) Pre-germination
b) Before the foliage closes.
   7. No treatments were given to the crop against the carrot fly vector, Psila rosae.

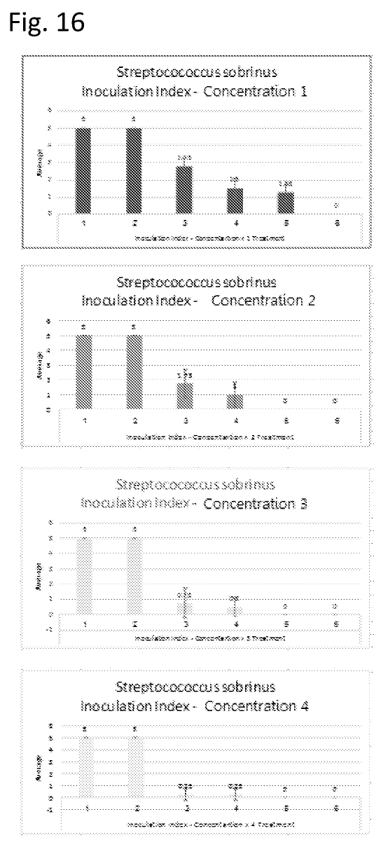

The trial map used is shown in FIG. 18. As indicated in this field map, two different treatments (T1 and T2) and a control group were used. Each test treatment consisted of the administration of two different types of perlite granule:

a) Granules soaked in an emulsified mixture of activating agents, said mixture comprising the activating agents shown in Table XII:

TABLE XII

| | Volume of each agent |
|---|---|
| | present in 250 ml of |
| Activating agent solution | emulsified mixture |
| Sclareol 98% | 1.94 |
| Nootkatone 98% | 1.94 |
| CBD 3% | 1.94 |
| Naringen 98% | 1.94 |
| Stevia 6% | 3.26 |

The soaked granules are then dried and coated (by spraying) with a Hydroxypropyl methylcellulose (HPMC) control-release polymer. The two different treatments (T1 and T2) differ with regard to the polymer concentration of the prepared granules:
T1: 10% (w/w)
T2: 20% (w/w)
   b) Granules soaked in B. subtilis (1.25 g Serenade® powder mixed into 500 ml water). Following drying, the granules are then spray-coated with the same control-release polymer as the granules containing the activating agents described above. For each of the two treatment regimens used, the B. subtilis granules had the following polymer concentrations:
T1: 5% (w/w)
T2: 10% (w/w)

In each treatment row, 20 granules of the granules containing the activating agents, and 20 granules containing B. subtilis ((a) and (b), above, respectively were added to each 1 m row, in triplicate (i.e. 20×3=60 of each granule type).

The various treatments used in the field study is summarized in Table XIII:

TABLE XIII

| | Control- | | |
|---|---|---|---|
| Treatment | release polymer | Granules/ | Granules |
| Group | concentration | m/row | g/1000 m² |
| T1: activating | 10% | 20 | 600 |
| agent granules | | | |
| T1: B. subtilis | 5% | 20 | 600 |
| granules | | | |
| T2: activating | 20% | 20 | 600 |
| agent granules | | | |
| T2: B. subtilis | 10% | 20 | 600 |
| granules | | | |

The control rows were treated with Perlite granules that did not contain either the treatment substances (activating agents or B. subtilis) or control-release coating.

### Results:

The condition of both the plant foliage and the developing carrots were monitored throughout the trial period.

### Foliage:

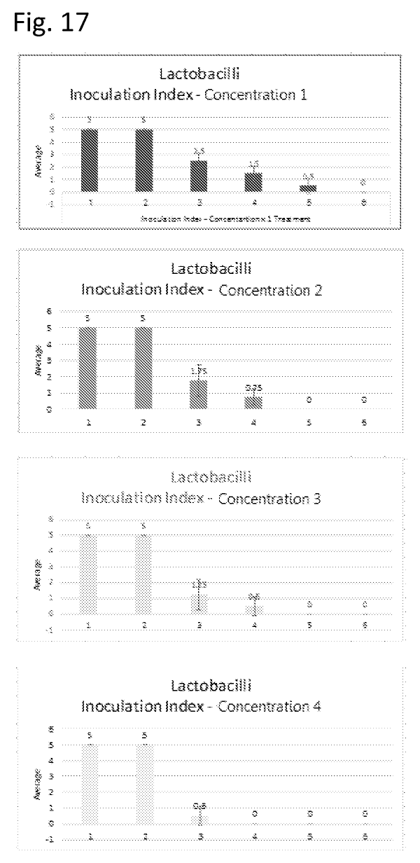

The percentage of plants in each of the three groups (control, treatment T1, treatment T2) that were adversely affected by the presence of Candidatus Liberibacter were assessed five months after sowing, and the average results calculated and shown in FIG. 19. It may be seen from this figure that treatment regimen T2 caused a marked decrease in the percentage of plants with adversely affected foliage (from 40.6% in control plants to 26.1% in the T2 group). This difference was found to be statistically significant (p<0.05).

### Carrots:

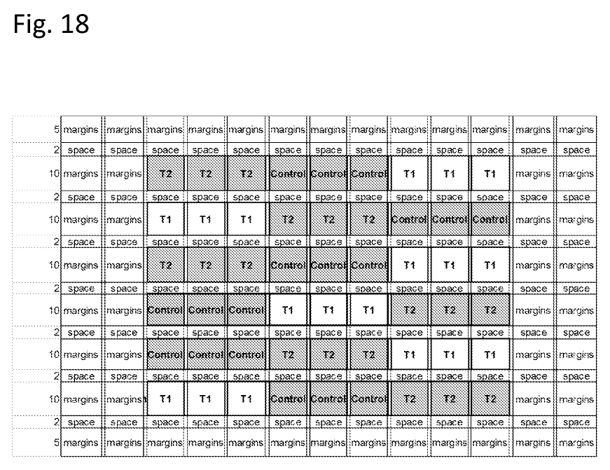

The average number of carrots adversely affected by the presence of the Candidatus Liberibacter infection was calculated for each of the three groups. As may be seen from FIG. 20, the average number of affected carrots was significantly reduced by both the T1 and T2 regimens, when compared with the control group.

### CONCLUSION

This preliminary study demonstrates that treatment of growing carrot plants with a granulated formulation containing a composition of the present invention significantly reduces the number of both plants and carrots which contain lesions caused by Candidatus Liberibacter infection.

### Example 16

### Preliminary Study of the Effect of a Composition of the Present Invention on the Survival of Honey Bees

### Introduction:

In recent years, a dramatic decline in the numbers of honey bees (mainly of the species Apis mellifera) has been observed. In view of the importance of the honey bee for agriculture and horticulture, this phenomenon (known as colony collapse disorder) has had severe consequences for those fields of activity. Colony collapse disorder is characterized by the disappearance of large numbers of worker bees in a colony, usually leaving behind the queen together with only a small number of nurse bees to tend the queen and the remaining immature bees. While the precise cause of colony collapse disorder is not conclusively known, it is likely that infection with viruses and/or fungi, possibly in conjunction with infestation by mites such as the Varroa mite (acting as a vector for viral infection), play a major role. It is known, for example, that the Varroa destructor mite is highly destructive to honey bee colonies, and that this is due at least in part to the viruses that may carry, including deformed wing virus and acute bee paralysis virus. Other viruses have also been implicated in this phenomenon, including the Israeli acute paralysis virus. Also, there is evidence that certain fungal species, such as Nosema apis and Nosema ceranae may be implicated in the pathogenic process leading to colony collapse disorder. Finally, at least one major study has found that combination of viral (iridescent virus type 6) and fungal (N. ceranae) agents are likely to be involved in the pathogenesis of this phenomenon.

In the present study, the inventors postulated that the anti-microbial compositions of the present invention, with their broad spectrum anti-microbial activity, may be able to arrest or prevent the loss of honey bees.

### Methods:

This study was performed using 7 pairs of beehives located in Caminomorisco in the Extremadura region in Spain. One hive of each pair (labeled as ‘Hive no.-A’) was treated with a composition of the present invention. The other hive (labelled as ‘Hive no.-B’ served as a control, since it was not treated with said composition, but rather continued to receive the routine conventional maintenance treatments that were routinely administered to the hives.

The composition used to treat the test (‘A’) hives comprises the following components:

### a) Oil Phase

| | |
|---|---|
| Sclareol 98% | 8.00 g |
| Nootkatone 98% | 16.00 g |
| CBD 3% | 16.00 g |
| ALDO MO, HLB = 2 | 30.61 g |
| MCT | 68.00 g |
| Ethanol | 12.25 g |

### b) Water Phase

| | |
|---|---|
| Tween 80 | 170.10 g |
| Naringin 98% | 8.00 g |
| Stevia 6% | 14.00 g |
| Water | 657.04 g |

(Total weight of two phases: 1000 g)

The two phases were mixed together, to form an emulsion.

300 g of the emulsion was then mixed together with 20 g of the B. subtilis preparation Serenade® suspended in 20 ml water. A further 80 ml of water was then added, yielding 400 ml of a treatment solution. This treatment solution was then divided into two lots: 300 ml for treatment of the bees, and 100 ml for treatment of the larvae.

The treatment solution was administered to the adult bees in the ‘A’ hives by adding 300 ml of said solution to the bee feed preparation (300 ml distilled water mixed with 850 ml honey).

The treatment solution was administered to the bee larvae in the ‘A’ hives by adding 100 ml of said solution to the larval feed preparation (480 g pollen mixed into 80 g of honey).

Each beehive contained 10 trays, each tray being fitted with rows of wells, some of which contained adult bees and others contained the bee larvae. At the beginning of the trial and then at 14, 28, 42 and 62 days thereafter, the number of adult bees and larvae in these trays were assessed, yielding a population index in order to ascertain the effect of the treatment on the bee population in the hives.

### Results:

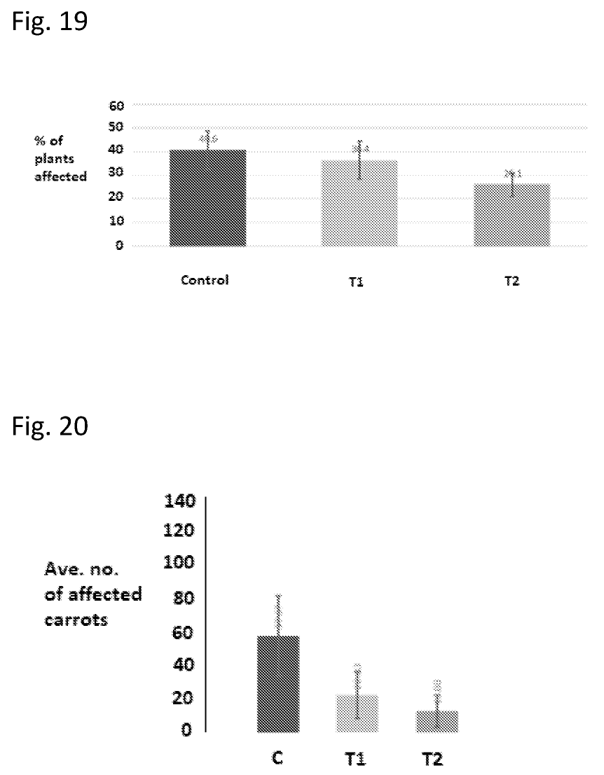

Excluding one pair of hives (5A and 5B, in which all of the colony died for other reasons), data on numbers of adult bees and larvae were obtained from 7 pairs of hives. The results are summarized in FIGS. 21 (larvae) and 22 (adult bees). It may be seen in these figures that treatment with the composition of the present invention (‘A’ Hives) resulted in the survival of larger numbers of both larvae and adult bees than in the untreated control beehives (‘B’ Hives).

It may thus be included that the mixture of B. subtilis and activating agents of the present invention is effective in preventing or reversing the reduction in honey bee populations.

### Example 17

### The Effect of a Composition of the Present Invention on Somatic Cell Count in Cow's Milk

### Introduction:

A high somatic cell count in cow's milk is used by the dairy industry as an indicator for potential infection and may disqualified the milk for consumption and use in food manufacture. A high somatic cell count may therefore lead to direct financial loss to the milk producer as result of the need to discard batches of milk. In addition, financial penalties may also have to be paid by the producer. Furthermore, in some regions, two reports of high somatic cell counts may lead to the distributors refusing to receive further milk supplies from the affected producer.

### Method:

Six cows identified with pre-existing elevated levels of somatic cells in the milk were each treated with one of the following topical gel preparations:

1. A gel containing 5% oil-water emulsion in a commercially available cream formula (Bio Spa enriched with wheat cream oil, manufactured by Sea of Spa, Arad, Israel) (2 cows).
2. A gel containing 5% oil-water emulsion in a commercially available Aloe-Vera cream (2 cows).
3. Cream-only control for preparation (1). (One cow.)
4. Cream-only control for preparation (2). (One cow.)

The emulsion containing the composition of the present invention was prepared as separate oil and water phases, which were then combined. The composition of the emulsion is given in the following table:

TABLE XIV

| | Ingredients | gram |
|---|---|---|
| oil phase | Sclareol 98% | 8.00 |
| | Nootkatone 98% | 16.00 |
| | CBD 3% | 16.00 |
| | ALDO MO, HLB = 2 | 30.61 |
| | MCT | 68.00 |
| | EtOH | 12.25 |
| Water phase | Tween 80 | 170.10 |
| | Naringin 98% | 8.00 |
| | Stevia 6% | 14.00 |
| | water | 657.04 |
| | | 342.96 |
| Emulsion | | 1000 |

0.5 g of Serenade® powder were added to 99.5 g of the above emulsion to create the treatment emulsion, which was then added at a concentration of 5% to each of the two cream preparations described hereinabove.

The treatment was done by covering the cows' teats with 5 ml of the gel immediately after milking.

The cows were milked and treated with the cream preparations twice a day.

Somatic cells samples were collected for analysis before starting the trial and after 15 days and 21 days.

### Results:

The numbers of somatic cells in the milk of each of the six cows included in the trial are shown in Table XV:

TABLE XV

| | Somatic | Somatic | Somatic | | |
|---|---|---|---|---|---|
| | Cells/liter | Cells/liter | Cells/liter | | |
| | Immediately | 15 days | 21 days | | |
| | before start | after start | after start | | |
| Cow # | of trial | of trial | of trial | Treatment | Notes |
| 145 | 4660 | 1509 | 50 | Emulsion + | |
| | | | | Bio Spa | |
| | | | | cream | |
| 114 | 3560 | 1050 | 40 | Emulsion + | |
| | | | | Bio Spa | |
| | | | | cream | |
| 253 | 1850 | 970 | 130 | Emulsion + | |
| | | | | Aloe Vera | |
| | | | | cream | |
| 989 | 4960 | 3850 | 1240 | Emulsion + | |
| | | | | Aloe Vera | |
| | | | | cream | |
| 901 | 1260 | 1850 | | Aloe Vera | stopped |
| | | | | cream- | |
| | | | | only control | |
| 974 | 1890 | 4230 | | Bio Spa | stopped |
| | | | | cream- | |
| | | | | only control | |

These results clearly show a dramatic reduction in the number of somatic cells in the milk samples taken from three of the four cows treated with the composition of the present invention. This reduction was particularly clear at 21 days from the start of the trial. In one of the cows treated with the composition of the present invention, (cow no. 989), while the results do not show such a dramatic change as in the other three test samples, there is nevertheless a significant decrease in the somatic cell account (to about 25% of the starting value.

These results indicate that the numbers of somatic cells in milk can be controlled using the composition of the present invention as prophylactic topical treatment in cattle and other milk-producing animals used in agriculture.

### Example 18

### Field Study: Direct Coating of Chickpea Seeds with a Composition of the Present Invention

### Introduction:

The normal sowing period for chickpeas (Cicer arietinum L) in Israel is at the beginning of February, mainly because of problems encountered with two soil-borne fungal pathogens—Ascochyta rabiei and Fusarium oxysporum f. sp. cicero. The existing chickpea varieties are unable to survive the Israeli winter as a result of the presence of these pathogens.

The purpose of this study was to investigate the effect of direct coating of chickpea seeds with a composition of the present invention, on the survival of chickpea plants

### Method:

Chickpeas were coated with emulsion A (a composition according to the present invention—see below) and subsequently coated with a controlled release polymer (E603 from Sekisui Specialty Chemicals, Japan).

Emulsion A was prepared from the following components:

| | |
|---|---|
| Serenade ® | 0.021 grams |
| Sclareol | 0.034 grams |
| Naringin | 0.034 grams |
| Nootkatone | 0.034 grams |
| stevia | 0.003 grams |
| CBD | 0.001 grams |
| Twin 80 | 0.723 grams |
| Polyaldo | 0.130 grams |
| MCT | 0.289 grams |
| ETOH | 0.052 grams |
| water | 2.929 grams |

Three different lots of chickpea seeds (variety 13, average weight 0.5 g/seed) were then prepared:

### Lot A:

7000 chickpeas were coated with 1.25 g emulsion A (diluted to 40 ml with water), using a fluidized bed coater. Subsequently, the emulsion-coated seeds were coated with 425 g polymer E603.

### Lot B:

7000 chickpeas were treated as in Lot A, except that the amount of emulsion A used (prior to dilution to 40 ml with water) was 2.5 g. The emulsion-coated seeds were coated with the control-release polymer exactly as in Lot A.

### Control:

3000 chickpeas were left uncoated and were not treated in any other way.

The trial field (total area of 1000 sq. meter) which contained heavy soil and was situated in Nahalal, Israel, was sown on Sep. 15, 2016 with a total of 17000 seeds variety 13 (average weight per seed 0.5 g), allocated to the three different lots described above. This field was known, from prior experience, to have a high Fusarium loading.

### Results:

The success or otherwise of the various treatments was determined by measuring the chickpea yield of each treatment area, and then extrapolating that weight to Kg per Dunam (1000 m²). The results of the various treatments are summarized in the following table:

TABLE XVI

| | | | kg/cultivated | kg/1000 |
|---|---|---|---|---|
| | chickpeas | sqm | area | sqm |
| Lot A | 7000 | 0.41 | 158.5 | 385 |
| Lot B | 7000 | 0.41 | 218.2 | 530 |
| Untreated Control | 3000 | 0.18 | 27.4 | 155 |

It may be seen from these results that both of the lots containing the seeds coated with the composition of the present invention and the release-control coating (i.e. Lots A and B) resulted in an increased yield of chickpeas when compared with untreated control. The greatest increase in yield was seen with the Lot B seeds, which were coated with twice the amount of the composition of the present invention, in comparison with the Lot A seeds.

These results indicate that direct coating of agricultural seeds with a composition of the present invention can enable the developing seedlings to resist infection by pathogenic soil-borne micro-organisms. This effect appears to be dose-dependent.

### Example 19

### The Fungicidal and Bactericidal Activities of Various Activating Agents in Conjunction with a Mixture of Probiotic Bacteria

In this study, a mixture of probiotic antibacterial species was used in combination with one or more activating agents, in order to investigate whether such species have the same effects in combination with said activating agents as were seen by the present inventors when the bacterial species is B. subtilis, as reported hereinabove.

### Method:

In this study, the bacterial mixture used in combination with the activating agents is ‘Jarro Dophilus’, which is a commercial probiotic product distributed in Israel by Altman Health Ltd.

The bacterial content of this product is as shown in Table XVII:

TABLE XVII

| | Quantity |
|---|---|
| The bacterial species | (Million/capsule) |
| L. rhamnosus R0-11 | 880 |
| L. Casei R0-215 | 680 |
| L. Plantarum R0-1012 | 340 |
| L. helveticus (acidophilus) R0-52 | 880 |
| B. Longum (Documented strain morinaga) BB536 | 680 |
| B. breve R0-70 | 340 |
| Pediococcus Acidilactici RO-1001 | 870 |
| Lactococcus Lactis spp. lactis RO-1058 | 330 |
| Total | 5000 |

A concentration of 0.5% W/W of this probiotic product was used in place of the B. subtilis preparation used in the other Examples given hereinabove. Combinations of this probiotic mixture with up to five activating agents were tested as described in Example 1, hereinabove, at the same concentrations described in that example (concentrations 3 and 4). Thus, the only difference between this study and that reported in Example 1, hereinabove is the fact that B. subtilis is replaced by the mixture of probiotic bacteria.

### Results:

As may be seen in Table XVIIIA—C, below, the results obtained using this mixture of probiotic bacteria as the bacterial element of the composition were essentially the same as those obtained with the mixtures containing B. subtilis that were reported in Example 1, hereinabove. The results shown in these table are for combinations containing activating agents used at concentration 4.

TABLE XVIIIA

| | | Fungal | Fungal | Fungal | Fungal | Mean |
|---|---|---|---|---|---|---|
| Test | | Index | Index | Index | Index | Fungal |
| No. | | rep 1 | rep 2 | rep 3 | rep 4 | Index: |
| 1 | Pathogen mix | 5 | 5 | 5 | 5 | 5.00 |
| 3 | 1 + 2 | 4 | 5 | 4 | 5 | 4.50 |
| 8 | 1 + 2 + 3 | 2 | 2 | 2 | 1 | 1.75 |
| 9 | 1 + 2 + 3 + 4 | 2 | 1 | 1 | 2 | 1.50 |
| 10 | 1 + 2 + 3 + 4 + 5 | 1 | 2 | 1 | 1 | 1.25 |
| 11 | 1 + 2 + 3 + 4 + | 1 | 1 | 1 | 1 | 1.00 |
| | 5 + 6 | | | | | |
| 12 | 1 + 2 + 3 + 4 + | 0 | 0 | 0 | 0 | 0.00 |
| | 5 + 6 + 7 | | | | | |

TABLE XVIIIB

| | | | | | | Mean |
|---|---|---|---|---|---|---|
| Test | | Bacterial | Bacterial | Bacterial | Bacterial | Bacterial |
| No. | | Index rep 1 | Index rep 2 | Index rep 3 | Index rep 4 | Index: |
| 1 | Pathogen mix | 5 | 5 | 5 | 5 | 5.00 |
| 3 | 1 + 2 | 4 | 5 | 4 | 4 | 4.25 |
| 8 | 1 + 2 + 3 | 1 | 2 | 1 | 2 | 1.50 |
| 9 | 1 + 2 + 3 + 4 | 1 | 1 | 2 | 1 | 1.25 |
| 10 | 1 + 2 + 3 + 4 + 5 | 1 | 1 | 1 | 2 | 1.25 |
| 11 | 1 + 2 + 3 + 4 + 5 + 6 | 1 | 0 | 0 | 2 | 0.75 |
| 12 | 1 + 2 + 3 + 4 + 5 + 6 + 7 | 0 | 0 | 0 | 0 | 0.00 |

TABLE XVIIIC

| | | JD | JD | JD | JD | Mean |
|---|---|---|---|---|---|---|
| Test | | Index | Index | Index | Index | JD |
| No. | | rep 1 | rep 2 | rep 3 | rep 4 | Index: |
| 1 | Pathogen mix | 0 | 0 | 0 | 0 | 0.00 |
| 3 | 1 + 2 | 1 | 2 | 2 | 1 | 1.50 |
| 8 | 1 + 2 + 3 | 2 | 2 | 3 | 2 | 2.25 |
| 9 | 1 + 2 + 3 + 4 | 3 | 3 | 2 | 3 | 2.75 |
| 10 | 1 + 2 + 3 + 4 + 5 | 4 | 3 | 3 | 3 | 3.25 |
| 11 | 1 + 2 + 3 + 4 + 5 + 6 | 4 | 4 | 3 | 4 | 3.75 |
| 12 | 1 + 2 + 3 + 4 + 5 + 6 + 7 | 5 | 5 | 5 | 5 | 5.00 |

(JD Index = Jarro Dophilus Index; equivalent to the B. subtilis index of Example 1.)

In conclusion: the mixture of probiotic bacteria used in this study appears to function in the same way as B. subtilis in the studies of anti-microbial activity reported hereinabove, and thus may replace said B. subtilis in compositions and methods of the present invention.

### Example 20

Maize seedling inoculation using various activating agents in conjunction with a mixture of probiotic bacteria In this study, maize seeds were inoculated in the same way as described in Example 10, hereinabove, except that the inoculation mixture (i.e. the composition of the present invention) comprised the probiotic mixture, ‘Jarro Dophilus’ described in Example 19, instead of B. subtilis.

### Results:

The following table presents the results for the inoculations carried out with the activation mixture at concentration 4:

TABLE XIX

| | Inoculation | Inoculation | Inoculation | Inoculation | Average |
|---|---|---|---|---|---|
| | Index Rep | Index Rep | Index Rep | Index Rep | Inoculation |
| Test mixture | 1 | 2 | 3 | 4 | Index: |
| 1 | 5 | 5 | 5 | 5 | 5.00 |
| 1 + 2-4 | 5 | 5 | 5 | 5 | 5.00 |
| 1 + 2-4 + 3-4 | 1 | 2 | 2 | 1 | 1.50 |
| 1 + 2-4 + 4-4 | 1 | 1 | 2 | 2 | 1.50 |
| 1 + 2-4 + 5-4 | 1 | 1 | 1 | 2 | 1.25 |
| 1 + 2-4 + 6-4 | 1 | 1 | 1 | 2 | 1.25 |
| 1 + 2-4 + 7-4 | 0 | 0 | 1 | 0 | 0.25 |
| 1 + 2-4 + 3-4 + 4-4 | 0 | 0 | 0 | 1 | 0.25 |
| 1 + 2-4 + 3-4 + 4-4 + | 0 | 1 | 0 | 0 | 0.25 |
| 5-4 | | | | | |
| 1 + 2-4 + 3-4 + 4-4 + | 0 | 0 | 0 | 1 | 0.25 |
| 5-4 + 6-4 | | | | | |
| 1 + 2-4 + 3-4 + 4-4 + | 0 | 0 | 0 | 0 | 0.00 |
| 5-4 + 6-4 + 7-4 | | | | | |

(Inoculation Index: semi-quantitative scale; 0 = healthy, 5 = dead).

It will be seen from these results that the probiotic mixture used in this study, when administered to the maize seeds in combination with the activating agents indicated in Table XIX, was able to protect the developing maize seedlings in much the same way, and to the same extent, as the composition in which the non-pathogenic bacterial species was B. subtilis (Example 10).

1. A method for preventing and/or treating infection of plant or animal host species by fungal, bacterial and/or viral pathogens, wherein said method comprises the steps of:
a) providing a mixture of one or more non-pathogenic bacteria and one or more activating agents; and
b) administering the mixture of step (a) to said host species.

2. A method for preventing and/or treating infection of plant or animal host species by fungal, bacterial and/or viral pathogens, wherein said method comprises the steps of:
a) providing separately:
(i) a composition comprising one or more non-pathogenic bacteria; and
(ii) a composition comprising one or more activating agents; and
b) separately administering each of compositions (i) and (ii) to said host species.

3. The method according to claim 1, wherein the non-pathogenic bacteria are selected from the group consisting of Bacillus subtilis and probiotic bacteria.

4. The method according to claim 3, wherein the non-pathogenic bacteria are bacteria of the species Bacillus subtilis.

5. The method according to claim 4, wherein the strain of Bacillus subtilis is the QST 713 strain.

6. The method according to claim 4, wherein the non-pathogenic bacteria are one or more species of probiotic bacteria.

7. The method according to claim 5, wherein the probiotic bacteria are selected from the group consisting of L. rhamnosus, L. Casei, L. Plantarum, L. helveticus (acidophilus), B. Longum, B. breve, Pediococcus Acidilactici, Lactococcus lactis and combinations thereof.

8. The method of claim 2, wherein the first composition to be administered to the host species is the composition comprising the non-pathogenic bacteria.

9. The method of claim 2, wherein the first composition to be administered to the host species is the composition comprising one or more activating agents.

10. The method of claim 2, wherein the composition comprising the non-pathogenic bacteria and the composition comprising the one more activating agents are administered to the host species at approximately the same time.

11. The method according to claim 1, wherein the one or more activating agents are substances having anti-inflammatory activity.

12. The method according to claim 11, wherein the one or more activating agents are substances capable of inhibiting the production of NO and/or TNF-a.

13. The method according to claim 12, wherein the activating agents each have an IC50 for the inhibition of NO production of less than 1.5 mg/ml and/or an IC₅₀ for the inhibition of TNF-a production of less than 2.5 mg/ml.

14. The method according to claim 12, wherein the activating agents each have an IC50 for the inhibition of NO production of less than 0.1 mg/ml and/or an IC50 for the inhibition of TNF-a production of less than 0.2 mg/ml.

15. The method according to claim 12, wherein the activating agents each have an IC50 for the inhibition of NO production of less than 0.05 mg/ml and/or an IC50 for the inhibition of TNF-a production of less than 0.1 mg/ml.

16. The method according to claim 1, wherein the one or more activating agents are selected from the group consisting of Sclareol, Naringin, Nootkatone, Steviol glycoside and cannabidiol.

17. The method according to claim 1, wherein the one or more activating agents are selected from the group consisting of Aster tataricus, Cyperus rotundus and combinations thereof.

18. The method according to claim 1, wherein the host species is a plant species.

19. The method according to claim 1, wherein the host species is an animal species.

20. The method according to claim 19, wherein the animal species is a domesticated or agricultural animal.

21. The method according to claim 19, wherein the animal species is an insect species of agricultural importance, particularly the honey bee.

22. The method according to claim 18, wherein the one or more non-pathogenic bacteria, one or more activating agents and/or combinations thereof are administered to the plant by means of foliar administration.

23. The method according to claim 18, wherein the one or more non-pathogenic bacteria, one or more activating agents and/or combinations thereof are administered to the plant by means of adding these substances to the medium in which said plant is growing.

24. The method according to claim 23, wherein the non-pathogenic bacteria, one or more activating agents and/or combinations thereof are administered in the form of granules coated with these substances.

25. The method according to claim 24, wherein the granules further comprise a release-control polymer.

26. The method according to claim 18, wherein the non-pathogenic bacteria, one or more activating agents and/or combinations thereof are administered by means of coating the seeds of the plant species with these substances prior to sowing said seeds.

27. The method according to claim 26, wherein the coated seeds further comprise a release-control polymer.

28. A composition comprising a mixture of non-pathogenic bacteria and one or more activating agents, wherein said one or more activating agents are substances having antiinflammatory activity.

29. The composition according to claim 28, wherein the activating agents each have an IC50 for the inhibition of NO production of less than 1.5 mg/ml and/or an IC50 for the inhibition of TNF-a production of less than 2.5 mg/ml.

30. The composition according to claim 28, wherein the activating agents each have an IC50 for the inhibition of NO production of less than 0.1 mg/ml and/or an IC50 for the inhibition of TNF-a production of less than 0.2 mg/ml.

31. The composition according to claim 28, wherein the activating agents each have an IC50 for the inhibition of NO production of less than 0.05 mg/ml and/or an IC50 for the inhibition of TNF-a production of less than 0.1 mg/ml.

32. The composition according to claim 28, further comprising one or more additional agents selected from the group consisting of stabilizers, solvents, sequestrants, emulsifier and release-control agents.

33. The composition according to claim 28, wherein the one or more activating agents are selected from the group consisting of Sclareol, Naringin, Nootkatone, Steviol glycoside and cannabidiol.

34. The composition according to claim 28, wherein the non-pathogenic bacteria are selected from the group consisting of Bacillus subtilis and probiotic bacteria.

35. The composition according to claim 34, wherein the non-pathogenic bacteria are bacteria of the species Bacillus subtilis.

36. The method composition according to claim 35, wherein the strain of Bacillus subtilis is the QST 713 strain.

37. The composition according to claim 34, wherein the non-pathogenic bacteria are one or more species of probiotic bacteria.

38. The composition according to claim 37, wherein the probiotic bacteria are selected from the group consisting of L. rhamnosus, L. Casei, L. Plantarum, L. helveticus (acidophilus), B. Longum, B. breve, Pediococcus Acidilactici, Lactococcus lactis and combinations thereof.

39. A composition comprising a mixture of one or more agents capable of causing activation of Bacillus subtilis, selected from the group consisting of Sclareol, Naringin, Nootkatone, Steviol glycoside and cannabidiol.

40. A method for increasing the yield of a plant of agricultural or horticultural importance by means of:
a) providing a mixture of one or more non-pathogenic bacteria and one or more activating agents; and
b) administering the mixture of step (a) to said host species.

41. A method for increasing the yield of a plant of agricultural or horticultural importance by means of:
a) providing separately:
(i) a composition comprising one or more non-pathogenic bacteria; and
(ii) a composition comprising one or more activating agents; and
b) separately administering each of compositions (i) and (ii) to said host species.

42. A method for increasing the yield of a product from an animal of agricultural importance by means of:
a) providing a mixture of one or more non-pathogenic bacteria and one or more activating agents; and
b) administering the mixture of step (a) to said host species.

43. A method for increasing the yield of a product from an animal of agricultural importance by means of:
a) providing separately:
(i) a composition comprising one or more non-pathogenic bacteria; and
(ii) a composition comprising one or more activating agents; and
b) separately administering each of compositions (i) and (ii) to said host species.

44. A method for increasing the ability of a plant or animal host species to resist damage caused by fungal, bacterial and/or viral pathogens comprising the steps of:
a) Providing a mixture of non-pathogenic bacteria and one or more activating agents; and
b) Administering the mixture of step (a) to a plant or animal host species.

45. A method for increasing the ability of a plant or animal host species to resist damage caused by fungal, bacterial and/or viral pathogens comprising the steps of:
a) providing separately:
(i) a composition comprising one or more non-pathogenic bacteria; and
(ii) a composition comprising one or more activating agents; and
b) separately administering each of compositions (i) and (ii) to said host species.

46. The method according to claim 40, wherein said method comprises preventing and/or treating infection of plant or animal host species by fungal, bacterial and/or viral pathogens, wherein said method comprises the steps of:
a) providing a mixture of one or more non-pathogenic bacteria and one or more activating agents, and
b) administering the mixture of step (a) to said host species, wherein the non-pathogenic bacteria are selected from the group consisting of Bacillus subtilis and probiotic bacteria.
